(19) United States
(12) Patent Application Publication
(10) Pub. No.: US 2023/0270678 A1
(43) Pub. Date: Aug. 31, 2023

(54) PHARMACEUTICAL COMPOSITIONS FOR IMPROVED DELIVERY OF THERAPEUTIC LIPOPHILIC ACTIVES

(71) Applicant: KARNAK TECHNOLOGIES, LLC, New York, NY (US)

(72) Inventor: Rafael Ezra, Tel-Aviv (IL)

(21) Appl. No.: 18/013,105

(22) PCT Filed: Jul. 29, 2021
(86) PCT No.: PCT/IL2021/050915

§ 371 Date: Dec. 27, 2022

Related U.S. Application Data
(60) Provisional application No. 63/058,266, filed on Jul. 29, 2020.

(51) Int. Cl. A61K 9/16; A61K 31/7048; A61K 9/00; A61K 9/107; A61K 31/07; A61K 31/00; A61K 9/70; A61K 47/32; A61K 31/05; A61K 9/48; A61K 36/53; A61K 31/593; A61K 31/01; A61K 47/44; A61K 9/51

(52) U.S. Cl. CPC A61K 9/1623; A61K 31/7048; A61K 9/0078; A61K 9/1075; A61K 31/07; A61K 31/658; A61K 9/7023; A61K 9/006; A61K 47/32; A61K 31/05; A61K 9/4891; A61K 36/53; A61K 31/593; A61K 31/01; A61K 47/44; A61K 9/5123

(57) ABSTRACT

A solid water-dispersible composition of matter comprising at least one sugar, at least one polysaccharide and at least one surfactant and at least one lipophilic active pharmaceutical ingredient (API), the composition comprises a plurality of micrometric particles each comprising a plurality of lipophilic nanospheres with an average size in the range of 50-900 nm, the at least one lipophilic API is contained in the micrometric particles and is distributed inside and/or outside the lipophilic nanospheres at predetermined proportions, thereby providing an improved delivery of the at least one lipophilic API. A sugar particle comprising a porous sugar material and lipophilic nanospheres having average sizes between 50-900 nm so that the lipophilic nanospheres are comprised within the porous sugar material, the sugar particle comprises at least one edible sugar, at least one edible oil, at least one edible polysaccharide, at least one edible surfactant and at least one lipophilic API.

### TECHNOLOGICAL FIELD

The invention relates to compositions and methods that increase bioavailability of therapeutic actives generally characterized as poorly water-soluble or lipophilic. The compositions and methods of the invention are designed and adapted for various routes of drug delivery and can be applicable to a wide range of poorly water-soluble drugs.

### BACKGROUND

Modern techniques for drug discovery such as high-throughput in vitro screening of receptor binding and combinatorial chemistry produce an increasing number of lipophilic, pharmacologically active compounds (APIs). The overall rate limiting factor in the oral absorption of these compounds is predominantly their solubility/dissolution in the hydrophilic intestinal milieu. According to the Biopharmaceutical Classification System (BCS), lipophilic drugs with low solubility and favorable permeability characteristics, would be classified as class II or IV compounds. Some notable examples are glibenclamide, bicalutamide, ezetimibe, aceclofenac, and amphotericin B, furosemide, acetazolamide, ritonavir, paclitaxel.

BCS Class II and IV compounds generally have low oral bioavailability, and as a result frequently fail to proceed to advanced stages of R&D. These types of compounds are not likely be good clinical candidates without accompanying development of special formulation methods to overcome problems of solubility or rate of dissolution. Various schemes have been developed to that end, but not without some major drawbacks.

Surfactants are routinely employed to increase the apparent aqueous solubility of poorly soluble drugs. Yet, the impact of micellar solubilization on the intestinal permeability of lipophilic drugs is still poorly understood. Many studies show that micellar formulations do not always retain their structure in the acidic pH of the stomach for the time required for their efficient adsorption. More recent research suggests that surfactants may have opposing effects on solubility of a given API and its subsequent intestinal membrane permeability.

Another popular approach to improve solubility relies on use of cyclodextrin-based formulations. Cyclodextrins are crystalline, non-hygroscopic, cyclic oligosaccharides with a hydrophilic outer surface and a lipophilic central cavity. From pharmaceutical point of view, cyclodextrins have gained widespread attention and use due to their ability to interact with poorly water-soluble drugs and increase their water solubility. However, a critical review of the literature reveals that cyclodextrins are not entirely predictable, and that their use may lead to counter-intuitive results and even reduction in the adsorption of some APIs.

Overall, for many solubility enhancers, there is a tradeoff between their tendency to improve solubility of lipophilic actives and their propensity to have negative effects on the respective intestinal membrane permeability of the same actives. In other words, a successful delivery method is conditioned on careful choice of solubility enhancer(s) and combinations of other excipients, and their cumulative impact on physicochemical and biological properties of the resulting formulations.

Therefore, there is a clear incentive for the development of new and more progressive formulations of lipophilic actives for overcoming the drawbacks of solubility/permeability tradeoff. Even more challenging would be to propose a general and more inclusive approach for improving bioavailability of various types of lipophilic actives.

There are numerous publications describing certain types of oral formulations with various lipophilic actives in the academic and patent literature, including those applying nanotechnology. It seems however that none of them is sufficiently inclusive and adaptable so as to be applicable to a wide range of pharmacologically relevant actives and to the processes of drug manufacturing.

Certain oral formulations with lipophilic actives were described in WO20035850, WO2015/171445, WO2016/147186, WO2016/135621 and WO2017/180954 with examples of cannabis, or isolated and pure cannabinoids, all of which are known for their lipophilicity. More general examples of formulations with lipophilic APIs using various nanotechnologies are provided in WO19162951 and WO14176389 as solid formulations, in WO2013/108254 as liquid formulations and in WO0245575 and WO03088894 with actives for specific uses in dentistry and cosmetics.

### General Description

The primary focus of this invention has been to explore novel strategies for improving the permeability and bioavailability of highly lipophilic drugs. Over the past years, disadvantages of conventional lipid-based formulations, such as physical instability, limited drug loading capacity, passive diffusion, active efflux in the GI tract and extensive liver metabolism, etc., have been extensively investigated. New lipidic formulations, and specifically the nanostructured lipid carriers, have been developed to overcome the barriers that lead to poor bioavailability of lipophilic drugs.

Nanotechnology is an area of rising attention that unwraps new possibilities for the pharma industry. Nanotechnology is superior to the conventional formulation technologies as regards capabilities to produce drugs with enhanced pharmacological characteristics, a better quality and safety, and increased shelf life. Today, nanomaterials serve as a basis for qualitative and quantitative production of old and new drugs with enhanced qualities and new types of functionalities.

With respect to poorly water-soluble or lipophilic actives, nano-delivery systems using specific solubility enhancers such as nanoemulsions, dendrimers, nano-micelles, solid lipid nanoparticles provide promising strategies for improving solubility, permeation, bio-accessibility, and oral bioavailability overall. Some of these systems further provide prolonged, and targeted delivery of actives.

The basic advantage of nanonization is in increasing the substrate surface area and dissolution rate. With lipophilic substances, nanonization can further increase saturation, solubility and reduce erratic absorption, thereby impacting on their transport through the GI wall and increasing their oral bioavailability. In addition, it has been reported that smaller particles are taken up more easily by macrophages, and thus provide a higher deposition rate and a better therapeutic index.

Nanoencapsulation of drug/small molecules in nanocarriers is a very promising approach for development of nanomedicine. Modern drug encapsulation methods allow efficient loading of drug molecules inside the nanocarriers, thereby reducing the drug-related systemic toxicity. Another application is targeting of nanocarriers to specific tissues and organs, and thus enhance the accumulation of the encapsulated drug at the diseased site. Nanoencapsulation can further protect drugs from premature degradation, and thus increase their stability in the circulation and tissues.

The present invention makes part of such emerging new technologies. The invention applies nanonization technologies to make and manipulate matter on a new size scale, and to create novel structures with highly unique properties and wide-ranging applications. To that end, the invention provides an exclusive formulation approach to resolving the specific problems of solubility and permeability related to lipophilic APIs, and to improving their bioavailability in vivo by oral and other non-invasive routes of administration. Importantly, as has been presently demonstrated, the formulation approaches of the invention are compatible with many kinds of lipophilic APIs, and therefore have the potential of wide-ranging pharmacological applications.

The compositions of the invention constitute a solid microparticulate matter which is fully dispersible in water. This quality, per se, constitutes a significant advantage in terms stability, storage, operability, and applicability to pharma. Other properties of the present compositions reside in the specific composition and arrangement of its core components, i.e., the sugars, the polysaccharides, the surfactants and the lipophilic nanospheres containing APIs in pharmaceutically acceptable oils or oil carriers. The present studies show that the oils and actives can be distributed inside and outside the lipophilic nanospheres, which is responsible for the feature of differential bioavailability characteristic of the compositions of the invention. The sugars, polysaccharides, and surfactants provide a formation or a porous mesh entrapping the lipophilic nanospheres. The formation or the porosity of the mesh can be modulated by the relative content of sugars, polysaccharides, surfactants, and oils, and the size of lipophilic nanospheres, which in turn impacts on the microparticulate structure and texture of the matter as a whole. Advantages of this particular structure have been revealed in surprising features of preservation of particles size upon dispersion in water, long-term stability, high loading capacity characteristic of the compositions of the invention.

Specific examples of the core components of the present compositions are trehalose, sucrose, mannitol, lactitol and lactose as sugars; maltodextrin and carboxymethyl cellulose (CMC) as polysaccharides; and ammonium glycyrrhizinate, pluronic F-127 and pluronic F-68 as surfactants. Regarding oil carriers, the compositions of the invention can use natural oils such as those enriched in monounsaturated fatty acids (MUFAs) and polyunsaturated fatty acids (PUFAs), e.g., Omega-3 and Omega-6, or synthetic oils, or mixtures of those.

Thus, the present compositions are essentially hybrid formulations combining the advantages of lipid-based formulations and nanoparticles in terms of high loading, long-term stability, reproducibility, enhanced bio-accessibility and oral bioavailability, and other properties. All these structural and functional properties of the present compositions been presently explored and exemplified.

More specifically, the key feature of preservation of the nanometric particle size upon reconstitution of the powder compositions in water was found to be consistent throughout various processes of production, storage conditions and various composition of sugars, oils, and actives, and upon fixation to films of polyvinyl alcohol (PVA) and even upon conversion of the compositions into the form of mist (EXAMPLES 1-2, 7, 9).

First, the feature of reproducible nanometric size of the lipophilic nanospheres is highly surprising, especially in view of the known tendency of the nanoemulsion to increase particle size or fuse under various conditions. Second, it is compatible with various administration modes which can involve drug dispersion and dilution. Third and the most important, it suggests that the benefits of nanonization can be preserved in the intestinal milieu, with the expected consequences of higher solubility, permeability, and bio-accessibility in situ.

Overall, it can be stated that the compositions of the invention provide consistent loading, entrapment, preservation, and reconstitution capacities of lipophilic actives that are preserved through various exposures, manipulations, and conditions.

The feature of high loading capability was further addressed in a study showing that the compositions of the invention can be loaded with APIs in oil carriers up to 90%-95% of the total weight (w/w), this is without disrupting the core characteristics of preservation nanometric size in the reconstituted powder (EXAMPLE 3).

The feature of chemical preservation of actives was addressed in a study showing that the compositions of the invention prevented degradation and oxidation of actives, even with actives sensitive to increased temperature, pro-oxidative species, and acidic pH such as lycopene and fish oil (EXAMPLE 2).

Another important feature of the compositions relates to different distributions APIs inside and outside the lipophilic nanospheres and the ability to increase the encapsulation capacity (EXAMPLES 1.6-1.7) This feature is highly useful in providing compositions with differential bioavailability for the entrapped and the non-entrapped APIs. This feature was further supported by finding in vivo of bi-phasic release profiles of actives in plasma and tissues characteristic of the compositions of the invention (EXAMPLE 4).

A biphasic release pattern provides an immediate burst of active release and further a prolonged active release. Animals exposed to the compositions of the invention have shown biphasic release profiles in plasma and tissues, while animals exposed to analogous lipid compositions showed only immediate release profiles. Due to limitations of the experimental time frame, the exact duration and nature of the prolonged release profiles (intermittent or sustained) remains to be established in future studies.

It can be stated that the immediate, prolonged, and potentially targeted release of actives are essential attributes of the present compositions, per se, as they arise from the specific composition and structure of their core components. Overall, these features are reflected in improved oral bioavailability of the present compositions over lipid forms with the same actives.

The concept of modulation of bioavailability is particularly applicable for actives which are meant to achieve therapeutic objectives. Modified release compositions provide chosen characteristics of time course and/or location of active-release and have the potential to achieve desired therapeutic outcomes. The final products can further include carriers, excipients, and various types of coating contributing to modified or targeted release of actives and providing the desired characteristics of consistency and taste to achieve better compliance.

Importantly, the compositions of the invention permit modulation of release profiles by controlling the distribution of APIs inside and outside the lipophilic nanospheres and thereby controlling the encapsulation capacity of APIs. Encapsulation of APIs is dependent on the amounts and types of oil carriers and/or the amount and types of sugars, polysaccharides, and surfactants. It can be further enhanced by removal of the non-encapsulated oil with hexane, for example.

In other words, the amount and/or the proportion of oil carriers and other components govern the structure and the entrapment capacity of the compositions regarding lipophilic APIs, which in turn governs their differential availability. Thus, the loading, encapsulation capacity and bioavailability of APIs can be modulated by varying the amounts and proportions of the core components of the compositions.

In practical terms, the compositions of the invention can include various distributions and ratios of APIs and oil carriers inside or outside the lipophilic nanospheres up to the extent of ratios between about 1:0 to 9:1, respectively, and specifically as ratios between about 4:1, 7:3, 3:2, 1:1, 3:7 or 1:4, respectively.

Another important feature of the present compositions resides in the fact that they are provided in a solid or semi-solid water dispersible form. Apart from the advantages in terms of stability and long-term storage, this feature is highly important when considering oral drug delivery. The oral route is the route of preference for drug delivery.

It has been further demonstrated that the present formulation approach is applicable to various types of sugars, oil carriers, combinations of oils and APIs, as single actives, and combinations of actives (EXAMPLES 1-11).

More specifically, it was demonstrated that the core properties of the present compositions were preserved during various processes of preparation, in other words, stemmed from the specific composition of core components and not from the process of preparation (EXAMPLE 1.5).

Further, the feature of uniformity and preservation of particles size remained consistent upon reconstitution of the compositions incorporated into polymeric films (EXAMPLE 7) and in solutions with high osmolarity mimicking the conditions of human skin (EXAMPLE 1.8). This combination of features makes the present compositions particularly attractive as a base for various dermal, and topical formulations.

In terms of biological properties, the feature of improved bioavailability has been demonstrated in two independent experiments in animal models, where the compositions of the invention exhibited advantageous patterns of immediate and prolonged release of actives into the circulation and tissues (EXAMPLES 4-5).

Further, the feature of improved bio-accessibility of actives, indicative of the effective amount of active remaining available for adsorption in the GI, was demonstrated for the compositions of the invention per se and was further enhanced in compositions incorporated in enteric-coated capsules (EXAMPLE 6). In other words, the compositions of the invention were found to be protective against gastric degradation of APIs.

Still further, the feature of improved permeability through various layers of the human skin was demonstrated in a set of experiments showing that the compositions of the invention had a significantly enhanced permeation though the 1St and 2nd layers of the stratum corneum compared to the respective oil forms and were related to a significantly higher rate of API penetration to the deeper layers of the skin (EXAMPLE 8).

The feature of exceptional adaptability and compatibility of the present compositions with various non-invasive modes of administration, apart from oral, has been presently demonstrated by incorporating the reconstituted powders into polymeric sublingual, dermal patches (EXAMPLE 7), and further, transforming them into the form of mist in an inhaler or nebulizer (EXAMPLE 9); all these, while preserving the core property of nanometric particle size.

More recent experiment with lipophilic antibiotics have shown that the powder compositions of the invention can enhance the efficacy of known lipophilic antibiotics against pathogenic bacteria, including highly resistant strains. Moreover, due to the unique properties of small particles size and improved solubility, they may have the ability to disrupt and/or enhance the permeability of antibiotic actives through microbial biofilm (EXAMPLE 10).

Overall, the present studies show that the powder compositions of the invention can protect APIs against various harmful exposures such as during production and storage and the acidic conditions in the GI, and further, can present APIs in a more bioavailable and bio-accessible forms to the circulation and tissues.

Thus, the presently proposed formulation approach offers a substantial degree of flexibility and applicability to various types of lipophilic APIs, or in other words, many of the therapeutic agents belonging to the groups of BCS Class II and IV compounds. Numerous drugs functioning as enzyme inhibitors, receptor antagonists and agonist, proton-pump and ion-channel inhibitors, inhibitors and reuptake inhibitors are classified as BCS Class II and IV.

A specific application is provided with incorporation of lipophilic APIs into micronized sugar particles of the invention. Specifically, the invention provides a smooth finely granulated sugar powder, which in itself is a composite particulate material made of a sugar crystalline matrix with entrapped lipophilic nanospheres. This structure confers to the composite the desired characteristics of sugar (e.g., taste, small crystals, larger surface area, higher solubility, mechanic, and thermodynamic stability, etc.) and the ability to entrap lipophilic APIs (EXAMPLE 10). This application is particularly advantageous for certain types of actives requiring taste masking.

Ultimately, the powder compositions of the invention have been related to properties of higher loading, higher encapsulation capacity, higher stability, modulated release and improved oral bioavailability and bio-accessibility of actives, which significantly exceeded those related to analogous lipid-based compositions; this, with a minimum concentration of surfactants. In addition, in contrast to lipid-based compositions where there is a limited play with excipients, the compositions of the invention permit application of a full range of excipients. All these make the compositions of the inventions a promising approach for improving the in vivo properties of lipophilic APIs, thus making them highly relevant for pharmaceutical and medical applications.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings.

FIG. 1 illustrates the feature of preservation of particle size characteristic of the powder compositions of the invention. Figure shows powder compositions comprising cannabinoids (THC or CBD) stored at 45° C. (oven) for 1, 35, 54, 72 and 82 days (3 months correlates to 24 months at RT).

FIG. 2 illustrates the feature of protection of lipophilic actives imparted by the present powder compositions. Figure shows TOTOX (overall oxidation state) values for fish oil (dashed) and the powder composition comprising the same (solid). Fish oil is sensitive to oxidation. Figure shows significantly lower levels of the primary and secondary oxidation products in the fish oil formulated into the powder composition starting from day 0 and up to day 14.

FIGS. 3A-3B illustrate the advantages of improved oral delivery and fast APIs release in plasma characteristic of the compositions of the invention (LL-P) compared to lipid-based compositions (LL-OIL) with CBD (3A) and THC (3B) as revealed after single oral dose administration in a rat model.

FIGS. 4A-4B reproduce these advantages in a controlled study comparing the powder compositions (LL-P) with CBD (4A) and THC (4B) and lipid-based compositions with the same APIs (LL-OIL). Figures show a specific bi-phasing active release profile in plasma characteristic of the compositions of the invention.

FIGS. 5A-5D show that the advantages of improved oral bioavailability are reproduced in tissues of animals administered with the powder compositions (LL-P) with THC and CBD and lipid-based compositions with the same APIs (LL-OIL). Figures show bi-phasing active release profile in the liver and brain characteristic of the compositions of the invention.

FIG. 6 shows that the advantages of improved oral delivery and bioavailability are applicable to a wide range of lipophilic actives. Figure shows actives release profile in plasma of the powder Vitamin D3 composition (solid) vs. the analogous lipid composition (dashed) upon single oral dose administration in a rat model. The powder composition shows a 2-fold increase in the concentration of Vitamin D3 over the lipid composition.

FIG. 7 illustrate the feature of enhanced bio-accessibility (degree of GI digestion) characteristic of the compositions of the invention using semi-dynamic in vitro digestion model. Figure show enhanced bio-accessibility of two APIs found in Oregano, Thymol and Carvacrol, of the powder compositions (P) compared to the respective oil forms (O), for each API and total APIs.

FIGS. 8A-8D further expand on the advantages of improved bio-accessibility using semi-dynamic model. Figures show that the protective effect and bio-accessibility of the powder composition can be further enhanced with enteric coated capsule (solid) compared the powder composition alone (dashed) and the oil-based composition (dotted). Figure relates to the bio-accessibility of total Thymol and Carvacrol (A), Carvacrol (B) and Thymol (C) at the end of the gastric phase, and the bio-accessibility of total Thymol and Carvacrol in the powder composition with enteric coated capsule (D) during the gastric and duodenal phases.

FIG. 9 illustrates the advantage of improved permeability through the full thickness of human skin as revealed in ex vivo model. Figure shows 6-fold increase in the permeability of Vitamin A in the powder composition compared to the lipid composition with the same API.

FIGS. 10A-10C show analogous experiment with respect to the permeability of CBD in the powder composition and lipid composition through the 1^{st} outermost layer of the stratum corneum (A), 2^{nd} layer of the stratum corneum (B), and a significantly higher cumulative transport of CBD into the deeper layers of the skin, overall (C).

FIGS. 11A-11B are SEM images (scanning electron microscope) under magnification ×1K (A) and ×5K (B) showing sugar particles with Theobroma oil with the characteristic smooth, finely granulated texture, and size in the range of 20-50 μm.

FIGS. 12A-12D illustrate the composite nature of the sugar particle of the invention. Figures are cryo-TEM images (cryogenic transmission electron microscopy) showing lipophilic nanospheres of average size of 80-150 nm entrapped in the sugar particle.

### DETAILED DESCRIPTION OF EMBODIMENTS

It should be appreciated that the invention is not limited to specific methods, and experimental conditions described herein, and that the terminology used herein is for the purpose of describing specific embodiments is not intended to be limiting.

Effective oral delivery of drugs is extremely influenced by aqueous solubility and intrinsic dissolution rate. Dissolution is the main rate-limiting step in the absorption of BCS Class II or IV drugs, together with additional factors such as hepatic first pass metabolism, drug efflux by P-gp, intra-enterocyte metabolism and chemical and enzymatic degradation.

When a poorly water-soluble drug enters the GI, a series of events limit its absorption: First, biliary secretions in the upper part of the GI play a role in the solubilization and emulsification of such drugs via formation of micelles, whereby it is presented to the absorptive membrane of the enterocyte in a more bio-accessible form. The capability of this process, however, is very limited and variable.

Second, the unstirred water layer (UWL), which separates the enterocytes brush border (atypical membrane) from the bulk fluid of the intestine, is a major hydrophilic barrier for the absorption of lipophilic compounds.

Third, in the enterocyte, there are biochemical barriers that affect drug absorption. CYP 3A4 (CYP3A4) enzymes in the enterocyte endoplasmic reticulum are responsible for a major part of drug metabolism in the intestinal wall. Studies have shown it to be a major barrier to the absorption of lipophilic drugs.

Four, drug efflux transporters located in the apical enterocyte membrane, such as P-gp, are also responsible for poor oral bioavailability of various drugs (e.g., digoxin, paclitaxel, doxorubicin, atorvastatin etc.). Apical P-gp efflux pumps, the most comprehensively studied transporters, reduce drug absorption by transporting the drug from the enterocyte back to the intestine. There is a link between CYP3A4 enzymes and the P-gp activities in working in concert to reduce the bio-accessibility of lipophilic drugs.

Five, after the intra-enterocyte metabolism, the P-gp efflux and before reaching the systemic circulation, the drug is transferred to the liver where it is exposed to various metabolic enzymes. This first pass hepatic metabolism is another significant barrier to the absorption of lipophilic drugs (e.g., β-blockers, calcium channel blockers and others).

Considering the above pharmacokinetic and pharmacodynamic obstacles, there is a pressing need in the design novel formulations increasing oral bioavailability of poorly water-soluble or lipophilic drugs.

Many researchers and pharma industries are developing various delivery systems basing on different nanoemulsion fabrication methods. One of the main disadvantages of nanoemulsions, in general, is their relative instability in terms of particles size over time. The nanoemulsions in solid powder forms, in particular, are known for lack of uniformity in particle size, and specifically after reconstitution in water. In addition, there is a general tendency to increase particle size due to fusion of particles under various conditions.

An increased particle size and lack of uniformity lead to significant variability in the absorption of substances entrapped in the nanoparticles, and poor oral bioavailability. Larger particles have a smaller surface area, and thus, an inferior absorption in plasma and tissues. Therefore, despite the potential of the nanoemulsion technology, there are still significant drawbacks with its incorporation into the pharma industry.

The present invention has proved to surpass these difficulties with nanonized powder compositions of lipophilic APIs, which while being readily dispersible in water preserve properties of loading, encapsulation and storage potential and improved oral bioavailability.

In the broadest sense, the compositions of the invention can be articulated as solid water-dispersible compositions of lipophilic active pharmaceutical ingredients (APIs). Importantly, due to the solid or semisolid constitution and the ability to produce homogenous dispersions in water, the present compositions are especially advantageous for long-term storage, preservation, and oral delivery, among others.

In numerous embodiments the compositions of the invention are provided a form of water-dispersible powders.

With respect to the actives, the term ‘active pharmaceutical ingredient (API)’ refers herein to any substance falling under the definition by WHO, i.e., substances intended to furnish pharmacological activity or to otherwise have direct effect in the diagnosis, cure, mitigation, treatment, or prevention of disease, or to have direct effect in restoring, correcting, or modifying physiological functions in human beings.

In numerous embodiments the compositions of the invention comprise one or more lipophilic API dissolved in an oil carrier or a pharmaceutically acceptable oil.

The term ‘lipophilic API’ requires additional attention. Lipophilicity refers to the ability of a chemical compound to dissolve in fats, oils, lipids, and non-polar solvents. Lipophilicity, hydrophobicity, and non-polarity describe the same tendency, although they are not synonymous. Lipophilicity of uncharged molecules can be estimated experimentally by methods measuring the partition coefficient (logP) in a water/oil biphasic system. For molecules that are weak acids or bases, the measurements must further consider the pH wherein the majority of species remain uncharged.

A positive value for logP denotes a higher concentration in the lipid phase.

Thus, in numerous embodiments, the invention applies to uncharged or weekly charged lipophilic API having a partition coefficient (logP) of more than 0.

More specifically, the invention is applicable to any lipophilic API with logP in the range between 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, or more.

The term ‘lipophilic API’ further relates certain classes of BCS drugs in relation to the four known categories by their solubility and permeability properties: Class I compounds with higher solubility and permeability; Class II with lower solubility but higher permeability; Class III with higher solubility but less permeability; and Class IV compounds with the lowest counts of solubility and permeability index.

Thus, in numerous embodiments, the compositions of the invention are particularly applicable to BCS Class II and IV compounds.

In some embodiments, the present compositions are applicable to BCS Class II compounds.

From another point of view, the compositions of the invention can be seen as a composite matter comprising a plurality of micrometric particles each comprising a plurality of lipophilic nanospheres with an average size in the range of about 50 nm to about 900 nm, the at least one lipophilic API is contained in the micrometric particles and is distributed inside and/or outside the lipophilic nanospheres at predetermined proportions, thereby providing improved delivery of the at least one lipophilic API.

In other words, the compositions of the invention are a solid particulate matter comprising particles at a micrometric scale, or particles with an average size in a range of between about 10-900 μm, or more specifically with an average size in the range of 10-100 μm, 100-200 μm, 200-300 μm, 300-400 μm, 400-500 μm, 500-600 μm, 600-700 μm, 700-800 μm and 800-900 μm.

In certain embodiments the powders of the invention can comprise particles with an average size in a range of between about 10 μm and to about 300 μm, or more specifically with an average size in the range of 10-50 μm, 50-100 μm, 100-150 μm, 150-200 μm and 250-300 μm.

The micrometric particles of the compositions of the invention, in themselves, are a composite matter comprising lipophilic nanospheres with an average size between about 50-900 nm, and more specifically, an average size in a range between about 50-100 nm, 100-150 nm, 150-200 nm, 200-250 nm, 250-300 nm, 300-350 nm, 350-400 nm, 400-450 nm, 450-500 nm, 500-550 nm, 550-600 nm, 650-700 nm, 700-750 nm, 750-800 nm, 800-850 nm, 850-900 nm and 900-1000 nm (herein an average size is an average diameter).

The size or diameter of the lipophilic nanospheres can be measured by DLS (dynamic light scattering) upon reconstitution of the powder composition in water, such measurements have been presently exemplified.

In numerous embodiments the size of the micrometric particles correlates to the size of the lipophilic nanospheres, meaning that the size of the lipophilic nanospheres governs the size of the of the micrometric particles.

The above implies that the lipophilic nanospheres are essentially entrapped in the micrometric particles. It further implies that this composite matter has certain porosity or arrangement permitting to contain the nanospheres. These two features have been presently exemplified. They are further reflected in the loading and the encapsulation capacity characteristic of the compositions of the invention (see below)

An important feature of the invention is that the shape and size of the lipophilic nanospheres are substantially maintained upon dispersion in water. In other words, due to particular composition and structure of the composite matter, the average size of the nanospheres remains unchanged under various conditions such as lyophilization, long-term storage, fixation and release from matrixes or films such as PVA, etc. The term ‘substantially maintained’ herein implies a deviation of 1-5%, 5-10%, 10-15%, 15-20% or up to 25% in average diameter before and after the manipulation or exposure to certain conditions.

An important feature of the present compositions resides in the distribution of the lipophilic APIs inside and outside the lipophilic nanospheres. This feature is responsible for the properties of immediate and/or prolonged delivery or of release of actives characteristic of the compositions of the invention.

In numerous embodiments the lipophilic APIs can be distributed inside or outside the lipophilic nanospheres at a ratio of between about 1:0 to 9:1, respectively.

In certain embodiments the lipophilic APIs can be distributed inside or outside the lipophilic nanospheres at a ratio of between about 4:1, 7:3, 3:2, respectively, meaning that they are present in an excess inside the lipophilic nanospheres.

In other embodiments the lipophilic APIs can be distributed inside or outside the lipophilic nanospheres at a ratio of between about 3:7 or 1:4, respectively, meaning that they are present in an excess outside the lipophilic nanospheres.

In still other embodiments the lipophilic APIs can be distributed inside or outside the lipophilic nanospheres at the ratio of about 1:1, meaning that they are present in approximately equal proportions inside and outside the lipophilic nanospheres.

The same feature can be further articulated in terms of encapsulation capacity of the lipophilic APIs into the compositions. The term ‘encapsulation capacity’ refers to the amount or a proportion of lipophilic APIs entrapped inside the particulate matter, or the powder composition as a whole.

In numerous embodiments the compositions of the invention can have an encapsulation capacity of lipophilic APIs up to at least about 80% (w/w) relative to total weigh, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to total weigh.

This feature is further related to loading capacity of the lipophilic APIs onto the compositions. The term ‘loading capacity’ refers to the amount or a proportion of lipophilic APIs that are loaded onto the powder composition.

In numerous embodiments the compositions of the invention can have a loading capacity of lipophilic APIs up to at least about 80% (w/w) relative to total weigh, or more specifically up to at least about 50%, 55%, 60%, 65%, 70%, 75%, 80%, 85%, 90%, 95%, 96%, 97% and 98% (w/w), or in the range between about 50%-98%, 60%-98%, 70-98%, 80-98% and 90-98% (w/w) relative to total weigh.

Another important feature characteristic of the present compositions is long-term stability or an extended shelf-life. This feature encompasses herein structural, chemical, and functional stabilities. In this instance, the structural stability is reflected in the ability to preserve particle size of the nanospheres upon reconstitution in water. The chemical stability reflects protection against degradation and oxidation under temperature, light and acidic pH, for example. The functional stability is reflected in preservation of properties of immediate and prolonged actives release.

In numerous embodiments the compositions of the invention can have a long-term stability of about at least about 1 year at room temperature, or more specifically up to at least about 6 months, 1 year, 2, years, 3 years, 4 years, 5 years at room temperature.

With respect to core components, in general, the compositions of the invention comprise at least one sugar, at least one polysaccharide and at least one surfactant and at least one lipophilic API.

In numerous embodiments the lipophilic APIs can be dissolved in at least one oil carrier or a pharmaceutically acceptable oil.

In other embodiments the lipophilic APIs in themselves can constitute an oily substance or a pharmaceutically acceptable oil.

Examples of booth these types of actives have been presently provided (EXAMPES 1-9).

As has been noted, the oil and the other core components are essentially responsible for the arrangement and porosity of the composite matter, and together with the oil component impact on the features of preservation of particle size, loading and encapsulation capacity characteristic of the present compositions.

The term ‘pharmaceutically acceptable oil’ encompasses herein to any oil that is generally safe, non-toxic, or biologically undesirable, and that which is acceptable for use in humans. The oils comprised in the compositions of the invention can be broadly characterized as non-toxic oils for food and pharmaceutical industry regulated by the FDA or EMA, or classified as GRAS (Generally Recognized As Safe).

In numerous embodiments the pharmaceutically acceptable oils can be obtained from a vegetable or an animal source, a synthetic oil or fat, or a mixture thereof.

In numerous embodiments the pharmaceutically acceptable oils can be natural oils, synthetic oils, modified natural oils, or combinations thereof.

In certain embodiments, the pharmaceutically acceptable oils can be selected from acylglycerols, mono- (MAG), di- (DAG) and triacylglycerols (TAG), medium-chain triglycerides (MCT), long chain triglycerides (LCT), saturated or unsaturated fatty acids.

In numerous embodiments the compositions of the invention can comprise pharmaceutically acceptable oils from plant or animal sources. For example, oils comprising a substantial proportion of monounsaturated fatty acids (MUFAs) and polyunsaturated fatty acids (PUFAs) would be advantageous in terms of additional health benefits.

In certain embodiments the pharmaceutically acceptable oils can be selected from the group of Omega oils, such as Omega 3, Omega 6, Omega 7 and Omega 10, or combinations thereof. Omega-3 and Omega 6 fatty acids play crucial role in brain function, normal growth and development. Omega-6 types help stimulate skin and hair growth, maintain bone health, regulate metabolism and reproductive system.

In certain embodiments, the pharmaceutically acceptable oil can be hemp oil, alone or in combination with other oils. Hemp oil contributes to skin regeneration.

Thus, from a broader perspective, the compositions of the invention can comprise any pharmaceutically acceptable type of vegetable oils, animal oils or fats, or essential oils. A nonlimiting list of relevant oils is provided in ANNEX A.

With respect to sugars, the sugars that are applicable to the present compositions can be broadly characterized as short chain carbohydrates and sugar alcohols, and more specifically oligo-, di-, monosaccharides and polyols. Sugars are safe and are ubiquitously used the pharmaceutical industry. The sugars can be from natural sources or synthetic.

In numerous embodiments the sugars can be selected from trehalose, sucrose, mannitol, lactitol and lactose.

In other embodiments the sugars can be xylitol, sorbitol, maltitol.

The relevant polysaccharides can be broadly characterized as polysaccharides suitable for use in pharmaceutical industry and generally considered as safe. They can be natural and/or synthetic polysaccharides. Specific examples of natural polysaccharides are fructans found in many grains and galactans found in vegetables, and further methyl-, carboxymethyl- and hydroxypropyl methyl-celluloses, and also pectin, starch, alginate. A nonlimiting list of relevant polysaccharides is provided in ANNEX A.

In numerous embodiments the polysaccharides can be selected from maltodextrin and carboxymethyl cellulose (CMC).

The relevant surfactants can be broadly characterized as non-toxic surfactants suitable for use in pharmaceutical industry, and specifically nonionic and anionic surfactants. Examples of anionic surfactants include (a) carboxylates: alkyl carboxylates-fatty acid salts; carboxylate fluoro surfactants, (b) sulfates: alkyl sulfates (e.g., sodium lauryl sulfate); alkyl ether sulfates (e.g., sodium laureth sulfate), (c) sulfonates: docusates (e.g., dioctyl sodium sulfosuccinate); alkyl benzene sulfonates, (d) phosphate esters: alkyl aryl ether phosphates; alkyl ether phosphates. Sodium lauryl sulphate BP (a mixture of sodium alkyl sulfates, mainly sodium dodecyl sulfate, C₁₂H₂₅SO₄⁻Na⁺). The non-ionic surfactant can include polyol esters, polyoxyethylene esters, poloxamers. Polyol esters include glycol and glycerol esters and sorbitan derivatives. Fatty acid esters of sorbitan (Spans) and their ethoxylated derivatives (Tweens, e.g., Tween 20 or 80) are commonly used non-ionic surfactants. A nonlimiting list of relevant surfactants (or emulsifiers) is provided in ANNEX A.

The most frequently used surfactants in the pharmaceutical industry are Polysorbate 20 and 80, and Poloxamer 188 in a concentration range of 0.001% to 0.1%.

In numerous embodiments the surfactants can be selected from ammonium glycyrrhizinate, pluronic F-127 and pluronic F-68.

In other embodiments the surfactants can be selected from monoglycerides, diglycerines, glycolipids, lecithins, fatty alcohols, fatty acids or mixtures thereof.

In other embodiments the surfactants can be sucrose fatty acid esters (sugar ester).

In numerous embodiments the compositions of the invention can comprise any combination of the above component, with more than one agent from the above groups.

With respect to APIs, as has been noted, present compositions encompass a wide range of actives. The relevant APIs can be broadly classified on the basis of their functionality, e.g., enzyme inhibitors, receptor antagonists, agonists, proton-pump and ion-channel inhibitors and/or reuptake inhibitors. Examples of lipophilic APIs belonging to these groups are Angiotensin-Converting Enzyme (ACE) inhibitors used for the treatment of hypertension, Selective Serotonin Reuptake Inhibitors (SSRIs) used in a wide range of psychiatric contexts, and Retinoid X Receptor (RXR) agonists used for the treatment of cancer, all of which are highly lipophilic.

Alternatively, the relevant APIs can be classified as antibiotics, antifungal, antiviral drugs, neuroleptics, analgesics, hormones, anti-inflammatory drugs, non-steroidal anti-inflammatory drugs, anti-rheumatic, anticoagulant drugs, beta-blockers, diuretica, anti-hypertension drugs, anti-atherosclerosis and antidiabetic drugs, anti-asthmatic drugs, decongestants, cold medicines. Examples of are lipophilic agents from these groups are synthetic opioids such as Pethidine, nonsteroidal anti-inflammatory drugs (NSAID) such as Flurbiprofen and Ibuprofen, antibiotics such as Rifarnpicin which is highly lipophilic, and statins such as Torvastatin, Simvastatin, Lovastatin.

In other words, the main criterion for the selection of candidate APIs for the present compostions is lipophilicity. Thus, the candidate lipophilic APIs can be from one of more of the general drug categories defined by the FDA. A nonlimiting list of relevant groups of drugs is provided in ANNEX A.

It should be noted that the compositions of the invention are further applicable to other lipophilic actives such as nutraceuticals, vitamins, dietary supplements, nutrients, antioxidants, and others, which can be introduced into the composition together with the lipophilic APIs.

As has been noted, in numerous embodiments, pharmaceutically acceptable oil, per se, can be characterized as nutraceuticals, vitamins, dietary supplements, nutrients and antioxidants. Example of such oils are Omega oils and fish oil exemplified on this application.

More generally, in numerous embodiments the lipophilic APIs can constitute between about 10% to about 98% of the compositions of the invention (w/w), or more specifically between about 10%-20%, 20%-30%, 30%-40%, 40%-50%, 50%-60%, 60%-70%, 70%-80%, 80%-90% and 90%-98% of the present compositions (w/w), or up to about 10%, 20%, 30%, 40%, 50%, 60%, 70%, 80%, 90% and 98% of the present compositions (w/w).

On the other hand, in numerous embodiments the sugars can constitute between about 10% to about 90% of the compositions of the invention (w/w), or more specifically between about 10%-20%, 20%-30%, 30%-40%, 40%-50%, 50%-60%, 60%-70%, 70%-80%, and 80%-90% of the present compositions (w/w), or up to about 10%, 20%, 30%, 40%, 50%, 60%, 70%, 80%, 90% of the present compositions (w/w).

Further, in numerous embodiments the present compositions can further comprise carriers, excipients, and additives for purposes of color, taste, and specific consistencies. The terms ‘carriers and excipients’ encompass herein any inactive substances that serve as the vehicle or medium for APIs and oils comprised in the compositions.

Another important feature of the compositions of the invention is the ability to provide an improved delivery of lipophilic APIs. The term ‘improved delivery’ encompasses herein improved drug solubility, drug absorption or drug release by any pharmacokinetic or pharmacodynamic parameters to provide improved oral, topical, dermal and transdermal bioavailability or drug delivery via any other route.

The notion of improved delivery has been based on the findings of superior pharmacokinetic and pharmacodynamic properties of the present compositions in plasma and tissues, upon oral administration (EXAMPLES 4-5) and topical application (EXAMPLE 8).

The term ‘improved’ encompasses herein a change in a range of about 5-10%, 10-15%, 15-20%, 20-25%, 25-30%, 30-35%, 35-40%, 45-50%, 50-55%, 55-60%, 60-65%, 65-70%, 70-75%, 75-80%, 80-85%, 85-90%, 90-95%, 95-100% relative to oil forms with the same actives, or up to 2, 3, 4, 5, 6, 7, 8, 9, 10, 20, 30, 40, 50, 60, 70, 80, 90, 100 fold relative to oil forms with the same actives.

This term further encompasses any advantageous change in drug release, permeability or absorption patterns, including the ability to modulate these patterns such as those revealed in the present compositions.

Thus, in numerous embodiments the compositions of the invention can provide an immediate release of lipophilic APIs to one or more parts of the GI tract, plasma or one or more tissues.

The term ‘immediate release’ implies that the lipophilic API can be measured in the GI or plasma within a relatively short period of time, such as after 1, 10, 20, 30, 40, 50, 60 min from the oral administration. It further implies a burst or a temporary release of API in the GI or plasma. The term further applies to the levels of API in organs or tissues (although with a slightly delayed timing), such as within 10, 20, 30, 40, 50, 60, 70, 80, 90 min from the oral administration thereof via oral or any other route.

In other embodiments the compositions of the invention can provide a prolonged release of lipophilic APIs to a part of the GI tract, plasma and/or tissues.

The term ‘prolonged release’ implies that active is measured in the GI, plasma and tissues with a lag, such as after 30, 60, 90, 120 min from the oral administration, and persists in the GI, plasma and tissues for 2 h, 3 h, 4 h, 5 h, 6 h, 7 h, 8 h and more after the oral administration.

This term further encompasses situations wherein API increases continuously, or increases and reaches a plateau, and increases in one or more temporary bursts.

In other embodiments the compositions of the invention can provide a biphasic release comprising an immediate and a prolonged release of lipophilic APIs to a part of the GI tract, the plasma and/or tissues.

In certain embodiments the compositions of the invention provide immediate and/or prolonged release of lipophilic APIs to one or more tissues of the central nervous system (CNS).

In certain embodiments the compositions of the invention provide immediate and/or prolonged release of lipophilic APIs to lymphatic tissues, one or more part of the GI and/or the liver.

The feature of improved delivery of actives is directly related to improved oral bioavailability. Thus, in numerous embodiments the compositions of the invention provide improved oral bioavailability of lipophilic APIs compared to analogous oil forms. This feature has been presently exemplified with respect to various types of compositions of the invention.

Further, in numerous embodiments the compositions of the invention provide an improved bio-accessibility of lipophilic substances compared to analogous oil forms. The term ‘ bio-accessibility’ refers herein to an amount of API released in the GI tract and becoming available for adsorption (enters the bloodstream), it is further dependent on digestive transformations of API into a material ready for absorption, the absorption into intestinal epithelial cells and the pre-systemic, intestinal, and hepatic metabolism.

In numerous embodiments the compositions of the invention can further provide an improved permeation of lipophilic APIs into one or more part of the GI tract or one or more tissues compared to analogous oil forms.

Modulation of biological properties of a dug such as drug delivery, bioavailability, bio-accessibility and permeation can have significant impact on the potential to achieve desired therapeutic outcomes or better patient compliance.

More specifically, modulation of these properties can have significant impact on therapeutically effective dosing, the number of administration and the overall drug regimen.

The term ‘therapeutically effective amount’ (also pharmacologically, pharmaceutically, or physiologically effective amount) broadly relates to an amount of API needed to provide a desired level physiological or clinically measurable response. Analogous terms are ‘therapeutic dose’ or ‘therapeutically effective dose’ relate to doses of API in a pharmaceutical composition or a dosage form, which can produce an improvement/reduction of at least one symptom of a disorder, a disease or a condition.

With respect to the therapeutically effective doses, the present formulation approach provides an exceptional flexibility, and capacities of encapsulation and loading of various amounts of APIs. Due of the wide-ranging applicability of the present composition to various types of APIs, the effective amounts of can expressed by ways of proportions.

In numerous embodiments, the therapeutically effective amount of lipophilic APIs and other actives comprised in the compositions can be in the range between about between about 10% to about 98% of the composition (w/w), or more specifically between about 10%-20%, 20%-30%, 30%-40%, 40%-50%, 50%-60%, 60%-70%, 70%-80%, 80%-90% and 90%-98% of the present compositions (w/w), or up to about 10%, 20%, 30%, 40%, 50%, 60%, 70%, 80%, 90% and 98% of the present compositions (w/w).

Therapeutically effective amount or dose can be further expressed as a dose of API per single dosage form and/or per single administration, and further as a daily or a weekly dose implying multiple administrations.

With respect to therapeutic effect, an improvement or alleviation of symptoms o a disorder or a condition can be evaluated by one or more of the following parameters: a type and/or a number of symptoms, severity, frequency of symptoms, specific groups of symptoms (partial symptoms), and/or overall manifestation of symptoms in a subject or a group. The effect can be further expressed as a proportion of reduction on a severity scale, e.g., up to about 5%, 10%, 15%, 20%, 25%, 50%, 75%, 100% reduction of a symptom(s), or a complete abolition of symptom(s).

These parameters are further dependent on the specific API, and patient specific factors such pre-existing condition, compliance, etc. They imply estimates produced on individual basis (case-by-case) and estimates on population basis (clinical trial).

The mechanisms by which the compositions of the invention exert improved oral bioavailability are yet to be discovered. One can assume that the specific combination and structure of the core components and particles can contribute to one or more of the following mechanisms:
The lipophilic components can contribute to biliary secretion and emulsification of API. Various lipids have been shown to induce biliary secretion in the upper part of GI and enhance emulsification dependent drug absorption, thus improving bioavailability.
The nanospheres can facilitate the passage of API across UWL. It has been shown that the nanometric particle size improves the surface area, thereby improving the dissolution of hydrophobic drugs in UWL.
The encapsulation into nanoparticles protects API from enzymatic degradation. It has been shown that encapsulated drugs are less exposed to enzymatic degradation during absorption process and can stay for a longer time in the intestinal lumen in vivo.
It has been shown that certain lipid excipients and surfactants are capable of inhibiting P-gp-mediated drug efflux and have the potential to alter the pharmacokinetics profile of API in vivo.
Certain lipids and oils can further stimulate lymphatic transport, thereby providing a way to bypass the hepatic metabolism. Thus, for drugs that are extensively metabolized on the first pass through the liver, the lymphatic route can provide rescue and significantly enhance their bioavailability.

Thus, the presently proposed platform can provide a comprehensive and inclusive approach to design and development of novel formulations of lipophilic drugs with improved qualities of bioavailability, tissue distribution and short- and long-term effects.

The present compositions can be further characterized in terms of administration modes. In numerous embodiments the compositions of the invention can be adapted for oral, sublingual, or buccal administrations.

In other embodiments the compositions of the invention can be adapted for rectal, topical, dermal or transdermal administrations.

Yet in other embodiments the compositions of the invention can be adapted for inhalation or nebulization. These specific applications have been recently exemplified.

In numerous embodiments, the compositions can comprise coatings and package forms contributing to long term storage, stability, and other properties. Use of enteric-coated capsules and their role in enhancing the bio-accessibility of the compositions of the invention has been presently exemplified.

In numerous embodiments the compositions can comprise one or more carriers and/or one or more coatings.

Gastro-resistant and controlled release coatings are especially applicable to oral dose forms. Such coatings can be achieved by various known technologies, such as the use of poly(meth)acrylates or layering. A well-known example of poly(meth)acrylate coating is EUDRAGIT®. Apart from increasing actives effectiveness, poly(meth)acrylate coating further provides protection from external influences (moisture) or taste/odor masking to increase compliance.

The layering encompasses herein a range of technologies using substances applied in layers as a solution, suspension (suspension/solution layering) or powder (dry powder layering). Various characteristics can be achieved by use of supplementary materials.

It should be noted that certain types of coating can further enhance targeting to specific tissues and organs.

In other words, one of advantages of the present technology is its ability to provide a flexible product that can be adapted to various pharmaceutical technologies.

All the above further apply to the methods, dosage forms and a variety of other applications of the invention to pharmaceutical industry.

More specifically, it is another objective of the invention to provide a dosage form comprising a therapeutically effective amount of the compositions according to the above.

In numerous embodiments the oral dosage forms of the invention can be provided in the form of tablets or capsules.

Thus, in numerous embodiments the oral dosage forms of the invention can comprise a coating, a shell, or a capsule.

As has been noted, in numerous embodiments the coating, shell or capsule can contribute to the prolonged delivery of the lipophilic APIs.

In numerous embodiments the coating, shell or capsule contribute to enhanced bio-accessibility of the lipophilic APIs.

In numerous embodiments the dosage forms can comprise additional carriers, excipients, and other additives for purposes of color, taste and specific consistencies.

In numerous embodiments the dosage forms can be adapted for oral, sublingual, buccal, rectal, topical, dermal, or transdermal administrations.

In numerous embodiments the dosage forms can be adapted for inhalation or nebulization.

In certain embodiments the dosage forms can be in a form of sublingual, dermal or transdermal patches. Such patches using PVA plasticizing material have been presently exemplified.

For this specific application, the suitable plasticizing materials can be generally characterized as non-toxic water dissolvable materials. Specific examples can include but, are not limited to, synthetic resins such as polyvinyl acetate (PVAc) and sucrose esters and natural resins such as rosin esters (or ester gums), natural resins such as glycerol esters of partially hydrogenated rosins, glycerol esters of polymerised rosins, glycerol esters of partially dimerised rosins, glycerol esters of tally oil rosins, pentaerythritol esters of partially hydrogenated rosins, methyl esters of rosins, partially hydrogenated methyl esters of rosins and pentaerythritol esters of rosins. Also, synthetic resins such as terpene resins derived from alpha-pinene, beta-pinene, and/or d-limonene and natural terpene resins may be applied in the chewy base.

The invention can be further articulated by way of pharmaceutical compositions comprising the compositions according to the above, and optionally further comprising pharmaceutically acceptable carrier(s) and/or excipient(s).

From yet another point of view the invention provides a kit comprising one or more dosage form according to the above, and optionally further comprising a device for administering thereof.

In certain embodiments the kit of the invention can include an inhaler or a nebulizer. This application is particularly relevant to the compositions provided in the form of mist in the context of various pulmonary conditions such as asthma.

From another point of view, the invention provides compositions and dosage forms according to the above for use in improving the oral bioavailability of at least one lipophilic APIs comprised in the respective compositions or dosage forms.

From yet another point of view, the invention provides compositions and dosage forms according to the above for use in improving the bio-accessibility at least one lipophilic API comprised in the respective compositions or dosage forms.

Still from another point of view, the invention provides a series of methods for improving the oral bioavailability and/or the bio-accessibility of at least one lipophilic API for treating a disorder or a condition in a subject in need thereof, the main feature of such methods is administering to the subject therapeutically effective amounts of the compositions and dosage forms of the invention.

It is another objection of the invention to provide methods for treating or alleviating disorders or clinical or sub-clinical conditions that can be remedied by treatment with one or more lipophilic APIs. The main feature of such methods is administering to a subject in need thereof therapeutically effective amounts of compositions and dosage forms of the invention.

More specifically, the invention provides methods for treating or alleviating disorders that can be remedied by treatment with lipophilic API(s) in a subject in need thereof, wherein the method comprises administering to the subject a therapeutically effective amount of a solid water-dispersible composition of matter comprising at least one sugar, at least one polysaccharide and at least one surfactant and at least one lipophilic API, and wherein the composition comprises a plurality of micrometric particles each comprising a plurality of lipophilic nanospheres with an average size in the range of about 50 nm to about 900 nm, the at least one lipophilic API is contained in the micrometric particles and is distributed inside and/or outside the lipophilic nanospheres at predetermined proportions, thereby providing immediate and/or prolonged delivery of said lipophilic API (s).

In numerous embodiments said administering of lipophilic API(s) can be oral, sublingual, buccal, rectal, topical, dermal, and transdermal administering.

In other embodiments said administering of lipophilic API(s) can be via inhalation or nebulization.

In further embodiments said administering of lipophilic API(s) can further involve use of a device to facilitate the administering of the API(s).

In certain embodiments said administering of lipophilic API(s) can be made via a sublingual, dermal or transdermal patch of the invention.

In certain embodiments the methods of the invention can further comprise concomitant administering to the subject least one additional API, lipophilic or not lipophilic.

In numerous embodiments the additional lipophilic APIs can be provided in the compositions of the invention.

These aspects can be further articulated in terms of use of the above-described compositions in the manufacture of medicaments for treating or alleviating disorders or conditions that can be remedied by treatment with lipophilic API(s).

In numerous embodiments the invention provides use of the above-described compositions in the manufacture of medicaments having one or more lipophilic APIs with improved bioavailability and/or improved bio-accessibility.

In yet another aspect, the invention provides a method for making a composition with increased bioavailability and/or bio-accessibility of lipophilic API(s) by:
(i) mixing an aqueous phase comprising at least one sugar, at least one polysaccharide and at least one surfactant with an oil phase comprising at least one lipophilic API,
(ii) emulsifying the mix to obtain a nanoemulsion,
(iii) lyophilizing or spray dying the nanoemulsion.

In another aspect, the invention provides a method for increasing loading of at lipophilic API(s) contained in a composition by:
(i) mixing an aqueous phase comprising at least one sugar, at least one polysaccharide and at least one surfactant with an oil phase comprising at least one lipophilic API,
(ii) emulsifying the mix to obtain a nanoemulsion,
(iii) lyophilizing or spray dying the nanoemulsion.

A specific application of the present technology is to provide an especially attractive formulation of API(s) in a micronized sugar particle, which can be further incorporated into various foods, chocolates, and sweets.

Essentially, the invention provides a sugar particle comprising a porous sugar material and lipophilic nanospheres having average sizes between about 50 to about 900 nm so that the lipophilic nanospheres are comprised within the porous sugar material, the sugar particle further comprises at least one edible sugar, at least one edible oil, at least one edible polysaccharide, at least one edible surfactant and at least one API.

The term ‘porous sugar material’ is meant to convey a solid sieve-like material with voids or pores which are not occupied by the main structure of atoms of the solid material (e.g., sugar). This term encompasses herein a material with regularly or irregularly dispersed pores, and pores in the form of cavities, channels, or interstices, with different characteristics of pores size, arrangement, and shape, as well as porosity of the material as a whole (the ratio of pores volume vs. the volume of solid material) and composition of solid material.

As has been noted, in numerous embodiments the lipophilic nanospheres can have an average size in the range between about 50-900 nm, and specifically in the range between about 50-100 nm, 100-150 nm, 150-200 nm, 200-250 nm, 250-300 nm, 300-350 nm, 350-400 nm, 400-450 nm, 450-500 nm, 500-550 nm, 550-600 nm, 650-700 nm, 700-750 nm, 750-800 nm, 800-850 nm, 850-900 nm and 900-1000 nm.

In certain embodiments the lipophilic nanospheres can have an average size in the range between about 100-200 nm, and specifically in the range between about 100-110 nm, 110-120 nm, 120-130 nm, 130-140 nm, 140-150 nm, 150-160 nm, 160-170 nm, 170-180 nm, 180-190 nm and 190-200 nm.

In numerous embodiments the size of the sugar particles can be in the range between about 10 μm and about 300 μm, and specifically in the range between about 10-50 μm, 50-100 μm, 100-150 μm, 150-200 μm and 250-300 μm or more.

In certain embodiments the size of the sugar particles can be in the range between about 20 μm to about 50 μm, and specifically in the range between about 10-50 μm, 20-50 μm, 30-50 μm, and 40-50 μm, or up to at least about 20 μm, 30 μm, 40 μm, 50 μm.

Within the indicated size ranges, in numerous embodiments the sugar particles of the invention can have an irregular shape or form (EXAMPLE 11).

In numerous embodiments the edible sugars comprised in the sugar particles can be obtained from a vegetable or an animal source, a synthetic sugar, or a mixture thereof.

In further embodiments the edible sugars can be obtained from a sugar beet, a sugar cane, a sugar palm, a maple sap and/or a sweet sorghum.

In certain embodiments the edible sugars can be a mono- and/or di-saccharides selected from glucose, fructose, sucrose, lactose maltose, galactose, trehalose, mannitol, lactitol or a mixture thereof.

In numerous embodiments the edible sugars can constitute between bout 30% to about 80% of the sugar particle (w/w), or more specifically between about 20%-30%, 30%-40%, 40%-50%, 50%-60%, 60%-70%, 70%-80% and 80%-90% of the sugar particle (w/w).

In numerous embodiments the edible polysaccharides can be selected from maltodextrins and carboxymethyl celluloses (CMC).

In numerous embodiments the edible surfactants can be selected from ammonium glycyrrhizinate, pluronic F-127 and pluronic F-68.

In numerous embodiments the edible surfactants can be selected from one or more monoglycerides, diglycerines, glycolipids, lecithins, fatty alcohols, fatty acids.

In certain embodiments the edible surfactants can be sucrose fatty acid esters (sugar ester).

In numerous embodiments the edible oils can be obtained from a vegetable or an animal source, a synthetic oil or fat, or a mixture thereof.

In certain embodiments the edible oils can comprise Theobroma oil (cocoa butter).

In numerous embodiments the sugar particles of the invention can further comprise one or more food colorants, taste or aroma enhancers, taste maskers, food preservatives.

A nonlimiting list of substances applicable to this specific aspect is provided in ANNEX A.

Thus in this particular aspect, the invention can be perceived as a medical food containing one or more lipophilic APIs dispersed in a food matrix. This food matrix may be a traditional food type (such as a beverage, yogurt, or confectionary) or a nutritional fluid fed to a patient through a tube. A medical food is usually administered to treat a particular disease under medical supervision.

The invention further provides compositions and method of us for eradicating, preventing development and destruction of microbial biofilms. Compositions of the invention may be applied to any tissue or organ in a subject's body by any means disclosed herein to treat or prevent evolution of such biofilms. The biofilms may alternatively are such formed on a surface of a device or a tool such as those used in medical facilities.

The term “about” in all its appearances in the text denotes up to a ±10% deviation from the specified values and/or ranges, more specifically, up to ±1%, ±2%, ±3%, ±4%, ±5%, ±6%, ±7%, ±8%, ±9% or ±10% deviation therefrom.

### EXAMPLES

Any methods and materials similar or equivalent to those described herein can be used in the practice or testing of the present invention. Some embodiments of the invention will be now described by way of examples with reference to respective figures.

### Example 1: Physical Properties of the Powder Compositions

### 1.1 Preservation of Particle Size in the Reconstituted Compositions

Powder composition comprising 30% of AlaskaOmega (Omega 3) was prepared by nano-emulsification, freezing in liquid N₂ and lyophilization (48 h). Distribution and uniformity of the particle size was evaluated after nanoemulsification and lyophilization upon dispersion of the powder in TWD to 1% (w/w) using PDI (poly dispersity index) measured by DLS (dynamic light scattering). Measurements were perfumed in triplicates. PDI correlates to particle size. PDI values indicated that the nanoemulsion and the reconstituted powder contained a uniform and homogenous population of particles with the average size of 149 nm±SD and 190 nm±SD, respectively.

The results suggest that the particle size in the reconstituted powder compositions is relatively constant and preserved relative to the source nanoemulsions, and that this feature is uniform and homogeneous per sample, overall. The finding of preservation of particle size is further indicative of the same trend in saliva and the GI.

1.2 Preservation of Particle Size after Storage for 1 Month

The powders were stored for 1 month, and then reconstituted in TWD to 1% (w/w) or to 2% (w/w) and subjected to DLS or Cryo-TEM (transmission electron cryo-microscopy) analyses. The average particle size in the reconstituted powders was 218 nm±SD and 100 nm±S for DLS and Cryo-TEM, respectively, suggesting that the measurements are dependent on the technology.

Overall, the results suggest that the powder compositions are relatively stable and preserve that reconstitution capacity of a relatively uniform population of particles at a nanometric range.

1.3 Powder Compositions with Lycopene Oil and Hemp Oil

Powder compositions produced from lycopene oil and hemp (1:1.4, respectively) using the above method. DLS analysis was performed on the nanoemulsion and the reconstituted powder (1% w/w). DLS analysis showed a single population of particles in the nanoemulsion with the average size of about 590 nm, and two populations of particles in the reconstituted powder with the average size of about 272 nm and a minor peak at 79 nm. The particle size was not increased after lyophilization.

The results indicate that in terms of preservation and uniformity of particle size, the powder compositions with lycopene and hemp oil behave similarly to the powders with Omega 3. Overall, the results suggest that the technology is adaptable to various types of lipophilic drug carriers, i.e., oils and combinations of oils.

1.4 Preliminary Stability Studies in Compositions with Cannabinoids

Powder compositions with CBD or THC were stored at 45° C. (oven) for 1, 35, 54, 72 and 82 days (3 months correlates to 24 months at RT). Particle size was evaluated using DLS. The results are shown in Table 1 and FIG. 1.

TABLE 1
DS measurements in test samples

| Temp | AVG | PDI | PEAK |
|---|---|---|---|
| RT | 150.5 | 0.208 | 163.2 |
| 1 day at 45° C. | 149.1 | 0.213 | 151.9 |
| 35 days at 45° C. | 160.2 | 0.25 | 159.6 |
| 54 days at 45° C. | 150.1 | 0.216 | 144.7 |
| 72 days at 45° C. | 150.1 | 0.212 | 143.3 |
| 82 days at 45° C. | 153.7 | 0.205 | 154 |

AVG average diameter (nm)
PDI polydispersity index

The results show that the particle size was preserved for at least three months at 45° C., thus suggesting that the powder compositions have long-term stability and ability to preserve particle size upon reconstitution in water solutions, and most likely in the GI.

1.5 Compositions with Lactose and Hemp Oil

Nanoemulsions and the respective powders were prepared with lactose as a choice of sugar. The list of ingredients is detailed in Table 2.

TABLE 2
Specifications of test samples

| Lactose | 80% | 90% | 100% | 110% | 120% |
|---|---|---|---|---|---|
| Ammonium Gly | 3.05 | 3.05 | 3.05 | 3.05 | 3.05 |
| Meltodextrin | 13.68 | 13.68 | 13.68 | 13.68 | 13.68 |
| Lactose | 16 | 18 | 20 | 22 | 24 |
| Water | 145.74 | 145.74 | 145.74 | 145.74 | 145.74 |
| Hemp oil | 15.74 | 15.74 | 15.74 | 15.74 | 15.74 |

Nanoemulsions were prepared from lactose solution (80%) and maltodextrin (25-50° C.). Lactose was added to the mix to the concertation of 90%, 100%, 110%, 120% (relative to the initial concentration), together with Ammonium Gly and hemp oil. The nanoemulsions were homogenized by M-110EH-30 at 10,000-20,000 PSI (25-50° C.)×4 cycles. Powders were prepared using (1) lyophilization: freezing (−25° C. to −86° C.) and lyophilization (12-24 h, −51° C., 7.7 mbar); or (2) spray drying: using peristaltic pump (rate 8.5-20 g/min, air temp. 110-150° C., air flow 0.4-0.5 m³/min, atomizer pressure 0.15 MPa). DLS analysis of the reconstituted powders is shown in Table 3.

TABLE 3
DS measurements in tested samples

| Lactose | Drying | Yield | | Pump rate | Average Size |
|---|---|---|---|---|---|
| conc. | technology | (%) | T air out | (g/min) | (nm) |
| 80% | Spray dryer | 54.8 | 62 | 8.78 | 135.3 |
| 90% | Spray dryer | 63.8 | 62 | 9.66 | 127.6 |
| 100% | Spray dryer | 87.5 | 63 | 10.4 | 125.6 |
| 120% | Spray dryer | 87 | 63 | 10.1 | 124.6 |
| 80% | Lyophilizer | 100% | NR | NR | 136.1 |
| 100% | Lyophilizer | 100% | NR | NR | 127.8 |
| 110% | Lyophilizer | 100% | NR | NR | 125.4 |
| 120% | Lyophilizer | 100% | NR | NR | 124.5 |

The results point to preservation of particles size under various manipulations and concentrations of lactose. Overall, the results suggest that lactose can serve as an alternative sugar without disrupting the core properties of the compositions.

### 1.6 Loading Capacity and Distribution of the Lipophilic Component

Nanoemulsions were prepared with various types of oil carriers: Omega 7, TG400300, EE400300. Surface oil content was determined by hexane. Powders (5 g) were washed with hexane (50 ml), filtered, and washed (×4) with hexane (5 ml). Loss on drying (LOD) was performed on the filtrate under N₂ until stabilization of weight. The oil content inside the nanospheres was estimated as:
Omega 7-52.67%
TG400300-30.67%
EE400300-35.33%

The results suggest that up to about 50% lipophilic carrier can be incorporated into the lipophilic nanospheres, depending on the type of oil. A similar distribution can be assumed for the lipophilic API(s) dissolved in this and other types of carriers.

The results indicate that a substantial proportion of lipophilic carrier (and lipophilic API) can be present outside the nanospheres. This finding strongly supports the notion of differential bioavailability and biphasic release of the lipophilic APIs characteristic of the compositions of the invention.

More recent studies suggested that above 80% and 90% of lipophilic carriers and lipophilic APIs can be incorporated into the nanospheres.

Overall, these results are indicative of high loading capacity of lipophilic carriers and lipophilic APIs in the powder compositions of the invention.

### 1.7 Encapsulation Capacity of the Compositions

Encapsulation capability was estimated by the difference between the initial amount of API and the final amount unentrapped in the composition. Four different types of powders were prepared with the following lipophilic carriers/APIs using the above methods:
Vitamin D3 oil
Passionfruit oil
Medium-chain triglyceride (MCT) oil
Pomegranate seed oil

The non-encapsulated lipophilic carriers/APIs were removed with hexane (shaking 1 g powder in 10 ml n-Hexane for 2 min), the product was filtered and washed with hexane (×3), and the content of the entrapped lipophilic carriers/APIs was measured using Solvent extraction-gravimetric method. The results are shown in Table 4.

TABLE 4
The entrapped oil content of tested compositions

| | Before wash | After wash | Encapsulation |
|---|---|---|---|
| Oil/active | (gr/100 gr) | (gr/100 gr | efficiency |
| Vitamin D | 30.57 | 30.50 | 99.8% |
| Passion fruit | 30.31 | 29.46 | 97 2% |
| MCT | 29.06 | 28.79 | 99.1% |
| Pomegranate | 29.16 | 26.11 | 99.8% |

The results suggest a substantially highly loading capacity of lipophilic carriers/APIs into the compositions of the invention to the extent of about 97.0-99.8%.

### 1.8 Preservation of Particle Size in High Osmolarity Solutions

The feature of preservation of particle size was further studied in saline solution mimicking the osmolarity on the skin. To be compatible with the skin, topical formulations are expected to be stable and maintain their characteristic properties under the conditions of high salinity—typically 0.5-0.8% NaCl.

Nanometric powders were resuspended (1% w/w) in the saline solution (0.75% NaCl) and TDW. DLS analysis was performed as above. Tests were performed in triplicates. The raw data for the distribution of particle size is given below:
Water: Z Avg; 164.1 nm, pdi: 0.232, peak1: 175.3 (99.3%), peak2: 3508 (0.7%).
Saline Sol.: Z AVG; 158.2 nm, pdi: 0.236, peak1: 154.3 (98.6%), peak2: 4085 (1.4%).

The results show minor differences in particle size between the saline solution and water, 158 nm vs. 164 nm, respectively. The results indicate that the powder compositions retain their particle size and uniformity in high osmolarity solutions.

Preservation of nanometric size and larger surface area can provide deeper penetration of APIs into the skin and improved efficacy. Overall, this study suggests that the powder compositions of the invention have the potential to provide unproved preparations for topical delivery of therapeutic actives.

1.9 Compositions with Additional Lipophilic Carriers

Powders were prepared with various lipophilic carriers using the above methods:
Sample 1—Fish oil FO 1812 Ultra, 50% oil
Sample 2—KD-PUR 490330 TG90 Ultra, 30% oil
Sample 3—KD-PUR 490330 TG90 Ultra, 50% oil

Particle size was evaluated in the nanoemulsions and the reconstituted powders. The particle size remained surprisingly stable in the respective nanoemulsions and reconstituted powders, with an average size ranging from about 140-160 nm.

In summary, the different compositions showed consistency of particle size in the transition from nanoemulsion to solid forms. The particles size remained stable during the drying process, which is highly surprising in view of increased temperature and drying conditions. This experiment suggests high applicability of the technology for numerous types of lipophilic carriers and APIs.

### Example 2: Surprising Chemical Stability of Actives

2.1 Stability of Compositions with Cannabis Extracts

Cannabinoids are especially prone to chemical and photolytic degradation. Nanoemulsions were prepared with full spectrum Cannabis oil (50%) obtained from two Cannabis strains (THC or CBD enriched) and the other core components of the compositions of the invention.

The reconstituted powders yielded the characteristic particle size of about 150 nm and the expected cannabinoid spectrum in oil. Powders were stored in aluminum bags in 40° C. chamber under the following conditions:
1 gr per bag
O₂ scavenger
Silica humidifier

The experiment was performed in two independent runs for powders with THC and CBD enriched extracts (Powder A and Powder B) Cannabinoid analysis was performed using HPLC at Baseline (0), 30 days, 45 days, and 83 days (correlates to 10, 13, 24 months at RT). The results are shown in Tables 5 and 6.

TABLE 5
Cannabinoid analysis in Powder A

| Analyte content | | | | | Total |
|---|---|---|---|---|---|
| (% w/w) | THC-Δ-9 | CBD | CBG | CBN | cannabinoids |
| T0 | 2.71 | 1.05 | 0.09 | 0.08 | 3.93 |
| 10 months | 2.62 | 1.03 | 0.09 | 0.09 | 3.83 |
| 13 months | 2.68 | 1.02 | 0.07 | 0.09 | 3.86 |
| 24 months | 2.62 | 1.03 | 0.09 | 0.09 | 3.83 |

TABLE 6
Cannabinoid analysis in Powder B

| Analyte content | | | | | Total |
|---|---|---|---|---|---|
| (% w/w) | THC-Δ-9 | CBD | CBG | CBN | cannabinoids |
| T0 | 0.28 | 3.95 | 0.01 | 0.07 | 4.31 |
| 10 months | 0.28 | 3.98 | 0.01 | 0.02 | 4.29 |
| 13 months | 0.27 | 3.93 | 0.01 | 0.09 | 4.3 |
| 24 months | 0.28 | 3.98 | 0.01 | 0.02 | 4.29 |

The results indicate that the compositions of the invention provide long-term stability of APIs, cannabinoids and complex compositions of cannabinoids, for at least 24 months at RT. The recommended storage conditions should further include aluminum bags with 02 scavenger and/or moister desiccator.

Overall, under these conditions, the maximum degradation rate did not exceed 2.5% for the entire cannabinoid content and was even lower for specific cannabinoids (THC and CBD as CBN and CBG). This finding is further consistent with the content of CBN (in Powder A for example) as a known marker of cannabinoid degradation.

### 2.2 Stability of Compositions Comprising Lycopene

Carotenoids are sensitive to increased temperature, pro-oxidative species, and acidic pH. Nanoemulsions were prepared with lycopene oleoresin (6% lycopene w/w) and the other core components of the present compositions. Powders (4 gr) were heat-sealed with vacuum in aluminium bags with moister and oxygen scavengers, and stored for 0, 30, and 90 days at RT (25° C.), 4° C. and 40° C. (in duplicates). Products were tested by visual appearance, DLS and HPLC analyses at the indicated time points.

Visual analysis indicated that all samples preserved the typical confluence texture, and color during the storage period. DLS analysis indicated that the characteristic particle size of 225-272 nm was relatively preserved. The results are shown in Table 7. HPLC analysis showed minimal losses of lycopene during the storage period, i.e., 7%, 3%, and 1% for samples stored at RT, 4° C., and 40° C., respectively.

TABLE 7
DLS analysis of compositions with lycopene

| Storage temperature | Time 0 | Time 30 days | Time 90 days |
|---|---|---|---|
| RT (about 25° C.) | 260 nm | 225 nm | 236 nm |
| 4° C. | | 272 nm | 265 nm |
| 40° C. | | 246 nm | 251 nm |

Overall, the results suggest that the present compositions provide an extended shelf life for APIs such as lycopene and protect against their oxidation and degradation. Extended stability of 90 days at 40° C. corresponds to 2 years at RT. The recommended conditions should further include aluminum bags with moister and oxygen scavengers.

2.3 Stability of Compositions with Vitamin D3

Powders with vitamin D3 were stored at 40° C./RH 75° C. for 90 days. Vitamin D3 and ethoxy Vitamin D3 degradation products were detected by HPLC. Analytical tests were further validated by an external authorized laboratory (Eurofins). The results are shown in Table 8.

TABLE 8
HPLC analysis of compositions with vitamin D3

| | | Vitamin D | Eurofins |
|---|---|---|---|
| | Vitamin D | degradation product | results |
| Vitamin D3 oil | 24.14 mg/gr | 0.70 mg/gr | 26.3 mg/gr |
| Vitamin D3 powder | 6.76 mg/gr | 0.40 mg/gr | 7.7 mg/gr |
| Day 1 | | | |
| Vitamin D3 powder | 6.60 mg/gr | 0.47 mg/gr | Duplicate 1 - 6.9 mg/gr |
| Day 90 | | | Duplicate 2 -7.4 mg/gr |

Cholecalciferol tests were consistent with the certificate (1M iu/g). The results indicated that the encapsulated fraction contained 28%-29% Vitamin D3 compared to the 30% Vitamin D3 in the original oil preparation, suggesting minimal losses of active during the production process. Further, only minimal degradation was observed during the storage period (up to 5% API). The differences between duplicates can be explained by soldering. The powder had far fewer degradation products compared to the oil form. The study suggested potential stability of the powder form for a period of 2 years at RT.

The above studies suggest that the powder compositions of the invention have surprisingly long shelf-life and capability to preserve chemical stability of APIs. This feature is highly surprising, especially in view that the production process involves high pressure, water environment, both of which are unfavorable for lipophilic molecules, and further in view that reduction of particle size and increased surface area are expected to increase oxidation and chemical instability of actives. These findings further support pharmacological applicability of the present compositions and methods.

2.4 Stability in Compositions with Fish Oil

The protective property of the present powder compositions was further supported in a study using compositions with fish oil. Fish oils (60% Omega 3 fatty acids w/w) are known to oxidize readily by forming primary and secondary oxidation products.

Powder compositions were prepared from 40% fish oil (w/w) and the other core components. The oil and powder samples were exposed to environmental oxygen, heat-sealed with vacuum, and stored at 4° C. for 28 days. The primary (peroxide; PV) and the secondary (anisidine; AV) oxidation products were measured at days 0, 14, and 28. TOTOX value (overall oxidation state) was calculated using Formula:

TOTOX=AV+2*PV.

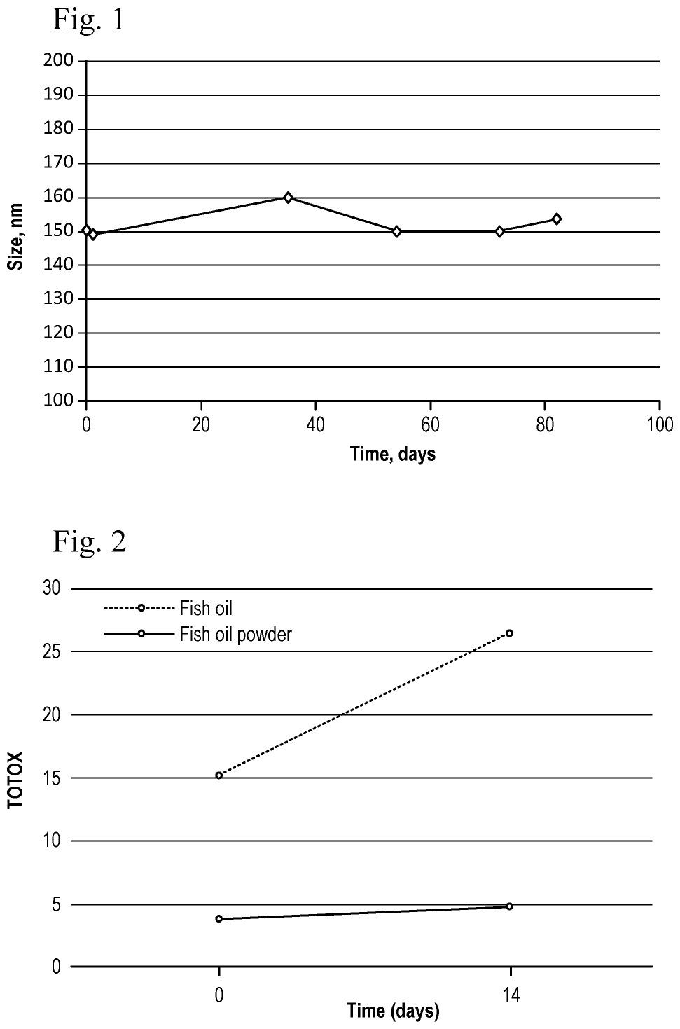

The results are shown in FIG. 2. The results show that the powder composition had a significantly lower TOTOX, i.e., a significantly lower concentrations of primary and secondary oxidation products, compared to the oil form starting from day 0 and up to day 14. The result of day 0 is further suggests that the production process of the powders does not lead to degradation, despite the exposure to water and oxygen.

Overall, the results support a surprising capacity of the powder compositions to protect actives and prevent their oxidation/degradation, most likely due to encapsulation. This property is further consistent with the previously shown long-term stability characteristic of the present compositions.

### Example 3: Surprising Loading Capacity

Loading capacity of the powder compositions was further studied in compositions containing concentrated Cannabis oil. Nanoemulsions were produced with raw RSO high THC concentrate (1gr) by the above methods. The nanoemulsions and the reconstituted powders yielded particles with the characteristic size of about 150 nm. The reconstituted powders were subjected to analysis of cannabinoids using HPLC. Table 9 shows the calculated vs. actual cannabinoid content.

TABLE 9
The measured and calculated THC content

| % w/w | Calculated | Measured |
|---|---|---|
| Δ9-THC | 8.945% | 8.45% |
| CBG | 0.276% | 0.24% |

The ratio between the calculated and actual content was 94.91%, and 86.9% for Δ9-THC and CBG, respectively, suggesting minimal losses of actives. The proportion of oil carrier relative to the total powder material further suggests a surprisingly high loading capacity of lipophilic carriers and APIs.

### Example 4: Surprising Oral Bioavailability of Cannabinoids

### 4.1 Pharmacokinetic Profiles in Plasma

Pharmacokinetic profiles (PK) of the present compositions were evaluated in a rat model. The study compared APIs release in plasma of two types of CBD/THC compositions: a powder composition of the invention (LL-P) and the analogous oil form (LL-OIL). The study used the following end points:
i. Mortality and morbidity monitoring—daily.
ii. Body weight monitoring—during acclimation and before dosing.
iii. Clinical observation—prior to and for 2 h after oral administration.
iv. Blood draws—at timepoints of 15, 30, 45, 60, 90, 120, 240, 420 min and 24 h.
v. Termination and organ collection (brain, liver) at 45, 60, 90, 120, 240 min.

The study used classical pharmacokinetic (PK) analyses in animals (N=18) divided into 6 groups (3 animals in each group).

Materials and Methods

Test item I: CBD/THC POWDER (LL-P): LL-CBD-THC 30% oil in powder.

Test item II: CBD/THC OIL (LL-OIL): LL-CBD-THC oil diluted in hemp oil.

Oral doses were prepared as follows: 150 mg LL-P was dissolved in 2.85 mg TDW; 45 mg LL-OIL was diluted in 1 ml hemp oil (per animal).

Male rats /18/376/456 g (sex/number/weight) were divided into groups (deviation of ±20% from mean weight in each group) and acclimatized (8 days). The study (1 cycle) was conducted in 6 groups (×3 animals and ×3 time points). Blood samples were collected at indicated time-points and stored. Group allocation is shown in Table 10. Animals were observed daily for toxic/adverse symptoms before and after administration. There were no findings of morbidity, pain, or distress during the entire study period.

TABLE 10
Group allocation

| Group | Test | Dose | Dose | | |
|---|---|---|---|---|---|
| (N) | Item | (mg/kg) | (ml/kg) | Route | Bleeding time point |
| 1M | LL-P | THC | 1 | Oral | 30, 90, |
| (N = 3) | | 13.5 | 0 | | 420 min |
| 2M | | CBD | 1 | | 15, 60, |
| (N = 3) | | 15.7 | 0 | | 240 min |
| 3M | | | 1 | | 0, 45, 120 |
| (N = 3) | | | 0 | | min, 24 h |
| 4M | LL-OIL | | 3 | | 30, 90, |
| (N = 3) | | | | | 420 min |
| 5M | | | 3 | | 15, 60, |
| (N = 3) | | | | | 240 min |
| 6M | | | 3 | | 45, 120, |
| (N = 3) | | | | | 24 h |

Results

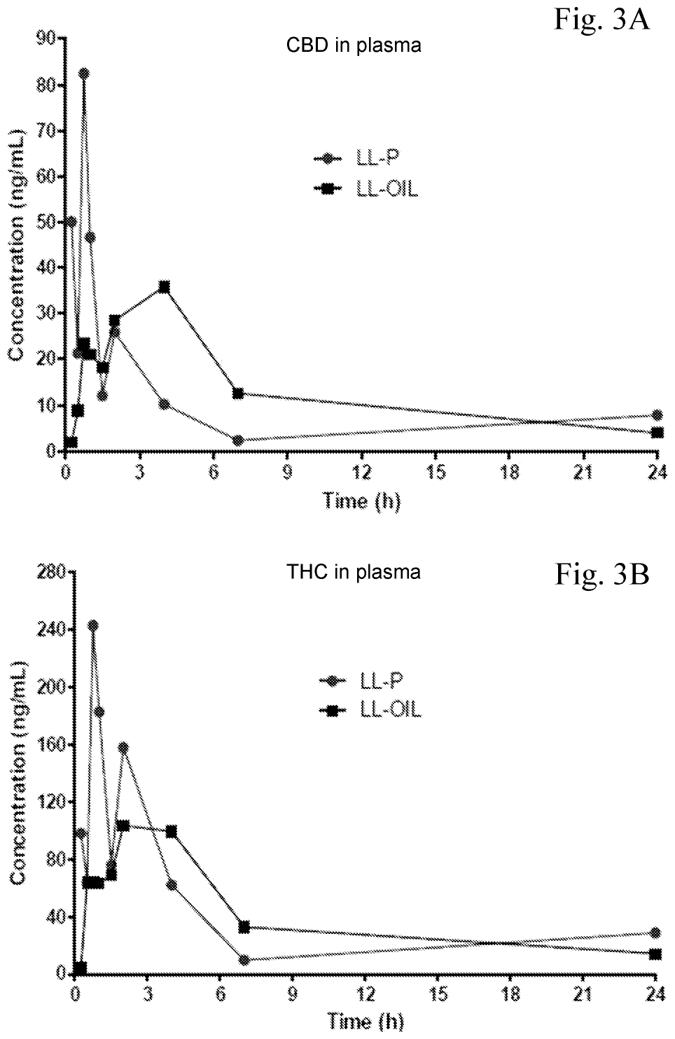

PK profiles of actives (CBD and THC) in plasma released from LL-P and LL-OIL are shown in Tables 11-12 (0-24 h and 0-7 h periods) and FIGS. 3A and 3B.

TABLE 11
PK analysis for 0-24 h

| Single Dose | | CBD | CBD | THC | THC |
|---|---|---|---|---|---|
| Parameters | | LL-P | LL-OIL | LL-P | LL-oil |
| Estimated Half-life | hr | 10.1 | 7.2 | 8.9 | 8.0 |
| Cₘₐₓ (obs) | ng/ml | 82.5 | 35.8 | 242.7 | 103.4 |
| Tₘₐₓ (obs) | hr | 0.75 | 4.0 | 0.75 | 2.0 |
| AUC₍₀₋₂₄₎ (obs area) | ng-hr/ml | 208.8 | 309.3 | 900.5 | 919.1 |

TABLE 12
PK analysis for 0-7 h

| Single Dose | | CBD | CBD | THC | THC |
|---|---|---|---|---|---|
| Parameters | | LL-P | LL-OIL | LL-P | LL-oil |
| Estimated Half-life | hr | 1.6 | 3.9 | 1.5 | 2.9 |
| Cₘₐₓ (obs) | ng/ml | 82.5 | 35.8 | 242.7 | 103.4 |
| Tₘₐₓ (obs) | hr | 0.75 | 4.0 | 0.75 | 2.0 |
| AUC₍₀₋₇₎ (obs area) | ng-hr/ml | 123.3 | 169.5 | 574.2 | 517.8 |

Conclusions

LL-P showed a significantly more rapid release profile in plasma compared to LL-OIL, both for CBD (Tmax 0.75 h vs. 4 h) and THC (Tmax 0.75 h vs. 2 h; LL-P and LL-OIL respectively). The observed plasma CBD Cmax was more than double (82.5 vs. 35.8 ng/mL, LL-P and LL-OIL respectively). The plasma THC Cmax was also significantly higher (242.7 vs. 103.4 ng/mL, LL-P and LL-OIL respectively). The oral bioavailability as reflected in calculations of AUC (area under curve) was about 40% higher for CBD in LL-oil than in LL-P, but was similar for THC in both forms.

### 4.2 Biodistribution in Tissues

Analogous study compared CBD/THC compositions in powder (LL-P) and oil (LL-OIL) forms with regard to release of APIs in plasma and selected organs (liver and brain). The study used the above end points, apart from:

iv. Blood draws—at timepoints of 0, 15, 30, 45, 60, 90, 120 and 240 min.

The study used classic PK analyses in animals (N=12) divided into 2 groups.

Materials and Methods

Test item I: CBD/THC POWDER (LL-P): LL-CBD-THC 30% oil in powder

Test item II: CBD/THC OIL (LL-OIL): LL-CBD-THC oil diluted in hemp oil

Oral doses were prepared as follows: 225 mg of LL-P was dissolved in 4.275 mg TDW; 67.5 mg LL-OIL was diluted in 1 ml hemp oil (per animal).

Male rats/12/376/456 g (sex/number/weight) were divided into groups (deviation of ±20% from mean weight in each group) and acclimatized (8 days). The study (1 cycle) was conducted in 2 groups (×6 animals, ×3-4 time points). Blood samples were collected at indicated time-points and stored. Organs (brain, liver) were collected after terminal bleeding and perfusion, and stored. Variations in organs weight were insignificant. Group allocation is shown in Table 13. There were no findings of morbidity, pain, or distress during the entire study period.

TABLE 13
Group allocation

| | | Dose | | | | | | |
|---|---|---|---|---|---|---|---|---|
| | Dose | volume | | | | | | |
| Group | (mg/kg) | (ml/kg) | Route | Animal | Bleeding time point | | Termination | |
| LL-P | THC | 10 | Oral | 1 | 0, 15, 45 | min | 45 | min |
| | 13.5 | | | 2 | 0, 30, 60, 240 | min | 240 | min |
| | CBD | | | 3 | 15, 45, 60 | min | 60 | min |
| | 15.7 | | | 4 | 30, 60, 90 | min | 90 | min |
| | | | | 5 | 45, 90, 120 | min | 120 | min |
| | | | | 6 | 0, 15, 30, 90 | min | 90 | min |
| LL- | | 3 | | 7 | 0, 15, 45 | min | 45 | min |
| OIL | | | | 8 | 0, 30, 60, 240 | min | 240 | min |
| | | | | 9 | 15, 45, 60 | min | 60 | min |
| | | | | 10 | 30, 60, 90 | min | 90 | min |
| | | | | 11 | 45, 90, 120 | min | 120 | min |
| | | | | 12 | 0, 15, 30, 90 | min | 90 | min |

Results

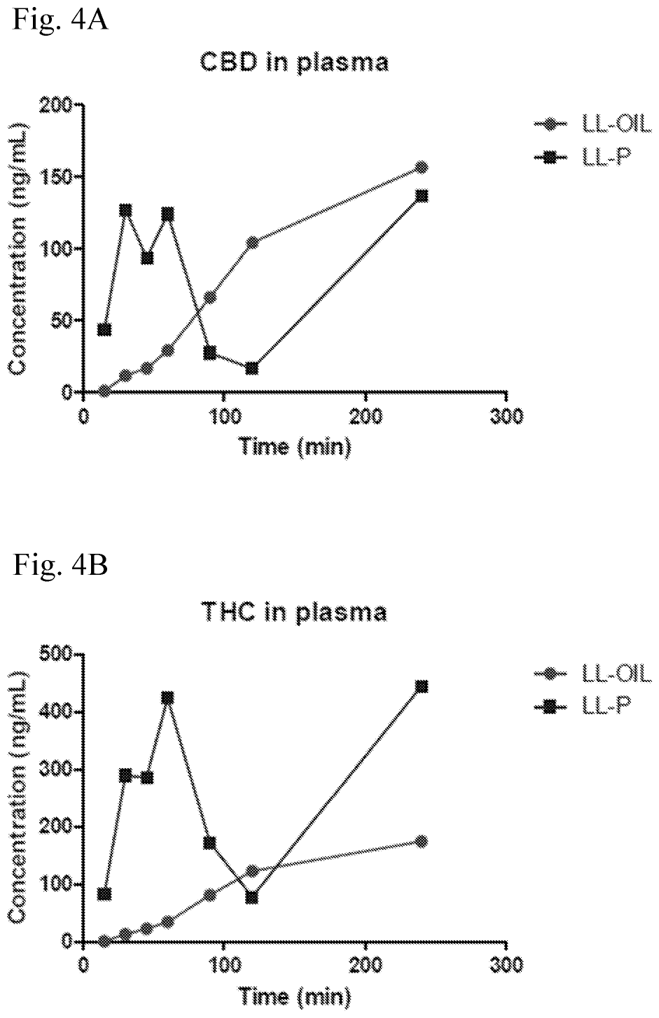

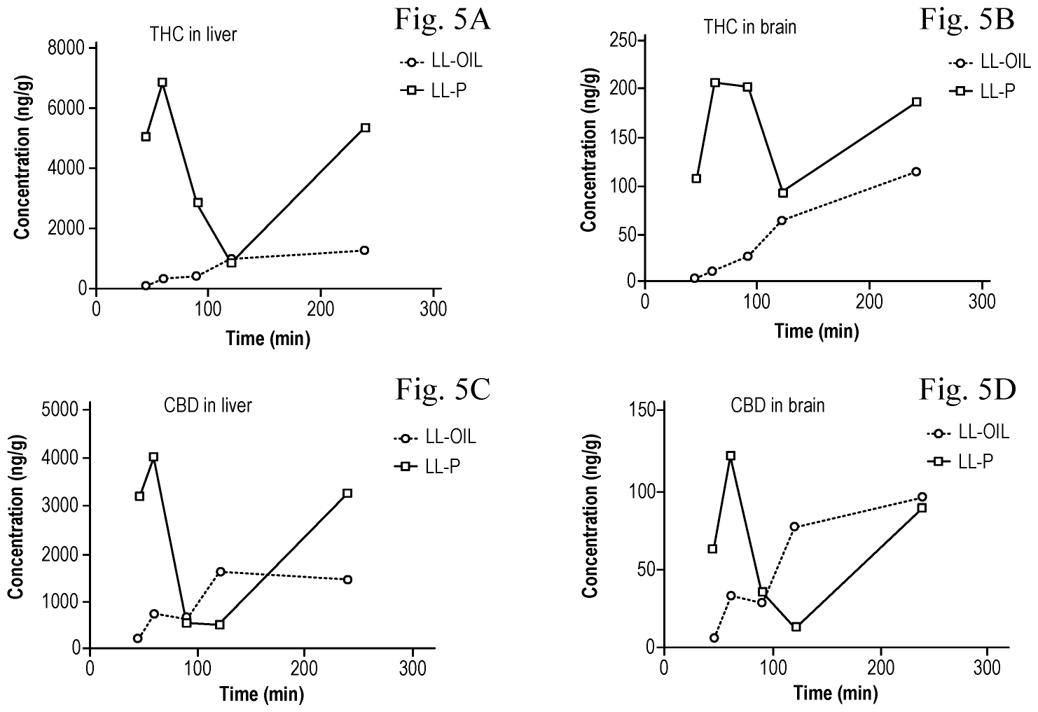

PK analyses of actives (CBD and THC) in plasma, brain and liver released from LL-P and LL-OIL are shown in Table 14, and FIGS. 4A-4B (plasma) and FIGS. 5A-5D (liver and brain).

TABLE 14
PK analysis of CBD and THC in plasma, brain and liver

| | | CBD | CBD | THC | THC |
|---|---|---|---|---|---|
| | | LL-P | LL-OIL | LL-P | LL-OIL |
| General PK parameters: | | PLASMA | PLASMA | PLASMA | PLASMA |
| Dose Amount | mg | 6.5 | 6.5 | 5.6 | 5.6 |
| Dosage | mg/kg | 15.7 | 15.7 | 13.5 | 13.5 |
| Cₘₐₓ (obs) | ng/ml | 137.0 | 156.6 | 444.4 | 174.6 |
| Tₘₐₓ (obs) | hr | 4.0 | 4.0 | 4.0 | 4.0 |
| AUC (0-4) (obs area) | ng-hr/ml | | | | |
| | | THC | THC | CBD | CBD |
| | | LL-P | BRAIN | LL-P | BRAIN |
| General PK parameters: | | BRAIN | LL-OIL | BRAIN | LL-OIL |
| Dose Amount | mg | 5.6 | 5.6 | 6.5 | 6.5 |
| Dosage | mg/kg | 13.5 | 13.5 | 15.7 | 15.7 |
| Cₘₐₓ (obs) | ng/g | 206.9 | 115.0 | 122.6 | 95.6 |
| Tₘₐₓ (obs) | hr | 1.0 | 4.0 | 1.0 | 4.0 |
| AUC(0-4) (obs area) | ng-hr/g | 536.5 | 215.0 | 201.0 | 221.5 |
| | | THC | THC | CBD | CBD |
| | | LL-P | LL-OIL | LL-P | LL-OIL |
| General PK parameters: | | LIVER | LIVER | LIVER | LIVER |
| Dose Amount | ng | 5.6 | 5.6 | 6.5 | 6.5 |
| Dosage | ng/kg | 13.5 | 13.5 | 15.7 | 15.7 |
| Cₘₐₓ (obs) | ng/g | 6828.8 | 1289.0 | 4037.2 | 1604.9 |
| Tₘₐₓ (obs) | hr | 1.0 | 4.0 | 1.0 | 2.0 |
| AUC(0-4) (obs area) | ng-hr/g | 12982.1 | 3004.1 | 7306.0 | 4184.4 |

Conclusions

In plasma, LL-P showed a biphasic release profile with an immediate increase of APIs during the first hour, followed by a decrease and another increase persisting until termination of the study period. In contrast, LL-OIL showed a monophasic release profile of actives during the study period (240 min).

The PK profiles in the liver and brain mimicked the plasma profiles. LL-P showed significantly more rapid permeation of both APIs into the tissues compared to LL-OIL, In the brain, CBD Cmax was higher in LL-P compared to LL-OIL (122.6 vs. 95.6 ng/g, respectively), the same was true for THC Cmax (206.9 vs. 115 ng/g, respectively). Similar results were observed in the liver.

These results suggest that the oral bioavailability of the LL-P compositions in plasma and tissues are superior to LL-OIL. Further, LL-P compositions can have additional advantage in providing a bi-phasic release profile combining immediate as well as prolonged actives release.

### Example 5: Bioavailability of Compositions with Vitamin D3

Advantageous oral bioavailability of the present compositions was further supported in a study comparing PK plasma profiles of compositions with vitamin D3 in powder and oil forms. Nanoemulsions were prepared as per standard protocol using both, lyophilization and spray drying. Table 15 shows that the powder compositions maintained the characteristic features of particle size, time to dissolution and others.

TABLE 15
QC test of the powder composition with vit. D3

| Vit. D powder | QC parameters | |
|---|---|---|
| Powder properties | Fine and white | |
| Vitamin D3 content % (w/w) | 300,000 | IU/g |
| Particle size-nm (in emulsion) | 150-200 | nm |

| | |
|---|---|
| Excipients | Disaccharide, polysaccharide, natural emulsifier |
| PH level in emulsion | 4.4 |
| Time to dissolution (sec) | <90 |
| Water content (%) | <2 |
| Flowability | Bulk density 0.5 gr/ml |
| | Tap density 0.7 gr/ml |
| | Angle of repose 45° |

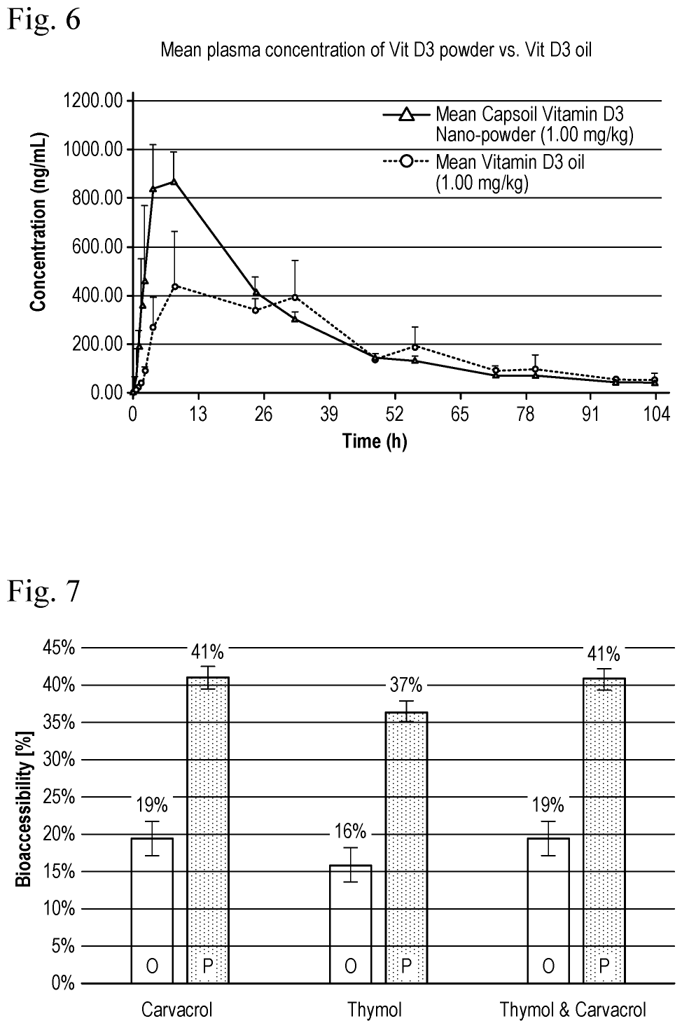

PK analyses were performed in rat plasma (N=9) upon administration of a single oral dose of cholecalciferol (1 mg/kg body weight). Blood samples were collected at 0, 0.25, 0.5, 1, 1.5, 2, 4, 8, 24, 32, 48, 56, 72, 80, 96 and 104 h (4 days). Steady-state cholecalciferol concentrations in plasma were measured by gas-liquid chromatography. Parameters were compared after subtraction of Baseline concentrations and using Baseline concentrations as a covariate. The results are shown in FIG. 6.

The results indicated that vitamin D3 in the powder composition peaked rapidly reaching at a double concertation in plasma relatively to the oil form, and further remained at a lower steady state concertation for at least 60 h (3 days). The bioavailability of vitamin D3 in the powder form as reflected in AUC (area under curve) was higher by 20%, and the half-life was longer by 15% (p<0.05) than in the oil form.

Overall, the results suggest improved oral bioavailability of lipophilic APIs in the powder compositions of the invention.

### Example 6: Enhanced Bio-Accessibility of Actives

### 6.1 Study In Vitro Mimicking the Conditions in the GI

The study explored the behavior of two actives, Thymol (2-isopropyl-5-methyl phenol) and Carvacrol (2-methyl5-(1-methylethyl) phenol), found in Oregano oil. Oregano oil is known for its beneficial properties, including antioxidant, free radical scavenging, anti-inflammatory, analgesic, antispasmodic, antibacterial, antifungal, antiseptic, and antitumor activities. Both these compounds have low solubility and permeability due to lipophilic properties and liability to degradation in the acidic condition in the stomach.

The study evaluated the bio-accessibility of Thymol and Carvacrol in the original oil form vs. the powder of the compositions of the invention using in vitro semi-dynamic digestion model. Bio-accessibility reflects the degree of GI digestion, i.e., an amount of compound released in the GI tract and becoming available for adsorption (e.g., enters the bloodstream). This parameter is further dependent on digestive transformation of the compound and its respective adsorption into intestinal cells and pre-systemic, intestinal, and hepatic metabolism. Bio-accessibility in vitro can be evaluated according to the following equation:

Bio-accessibility (%)=(Thymol and Carvacrol content after digestion in vitro/Thymol and Carvacrol initial content)×100

There are several types of in vitro digestion models: the static, semi-dynamic, and dynamic models. The static model is characterized by a single set of initial conditions (pH, concentration of enzymes, bile salts, etc.) for each part of the GI tract. It is relatively simplistic and has many advantages, but often provide a not realistic simulation of complex in vivo processes. The dynamic digestion model, in contrast, further includes corrections for geometry, biochemistry, and physical forces to better reflect in vivo digestion (e.g., continuous flow of the digestion content from the stomach to intestine, HCl addition, pepsin flow rate, gastric emptying, and controlled bile secretion). The semi-dynamic model is an intermediate model combining the advantages of both approaches. It includes pH modulation by HCl in the gastric phase and NH₄HCO₃ in the intestinal phase (unlike the static model) but has no continuous flow of the digestion contents and the intestinal stage begins after the gastric stage (unlike in the dynamic model).

Materials and Methods

APIs were tested in the forms of: (1) Oregano oil: 365 μl (˜300 mg Oregano oil) comprising 1.26 mg Thymol and 26.31 mg Carvacrol; and (2) Oregano powder: 1.11 gr the powder composition of the invention comprising 1.30 mg Thymol and 26.31 mg Carvacrol. The powder composition was produced according to the above method, yielding loading of 30% Oregano oil (w/w).

The two forms were tested in the semi-dynamic digestion system using INFOGEST protocol. The concentration of Thymol and Carvacrol was measured at the Baseline and after 2 h (representative of the end-gastric phase). Samples were analyzed by gas chromatography-mass spectrometry (GC-MS) using fused silica capillarity column (30 M, 0.25 mm), source temperature of 230° C., quad temperature of 150° C., and column oven temperature 250° C. for 3 min. Digesta sample (1 μl) was injected and concentration of analytes was calculated (peak area against standard peak area). The calibration curve showed linearity of the MS response. All preparations were analyzed by GC-MS before and after the in vitro gastric digestion at relevant time points. Chemical analysis of the oil and powder compositions was performed to assess loss of actives during powder preparation.

Results

Thymol and Carvacrol concentrations were reduced during the powder preparation process by 7% and 10%, respectively. In vitro digestions studies of the two forms showed that at the end of the gastric phase (2 h post-ingestion), the bio-accessibility of Carvacrol was 19% and 41% (more than twice) for the oil the powder forms, respectively. Similarly, the bio-accessibility of Thymol was 16% and 37% for the oil the powder forms. The bio-accessibility of both APIs was 19% and 41% for the oil and powder forms, respectively. In other words, while only about 20% APIs in the oil composition survived the acid pH in the stomach, the APIs survival in the powder composition was significantly increased. The results are shown in FIG. 7.

Conclusions

Overall, the results suggest that the powder compositions of the invention can protect actives from gastric degradation, and thereby increase their oral bioavailability and bio-accessibility to the circulation and tissues.

### 6.2 Comparative Study Including Powders in Enteric-Coated Capsules

Analogous study was performed, including the oil and powder forms as above and the powder form in enteric-coated capsules (acid resistant coating). Thymol and Carvacrol concentrations were measured at Baseline and after 2 h (end of gastric phase), with calculations of bio-accessibility as above. In addition, the powder in enteric-coated capsules was shifted from the stomach phase to the duodenal phase and tested after 4 h (end of duodenal phase).

Results

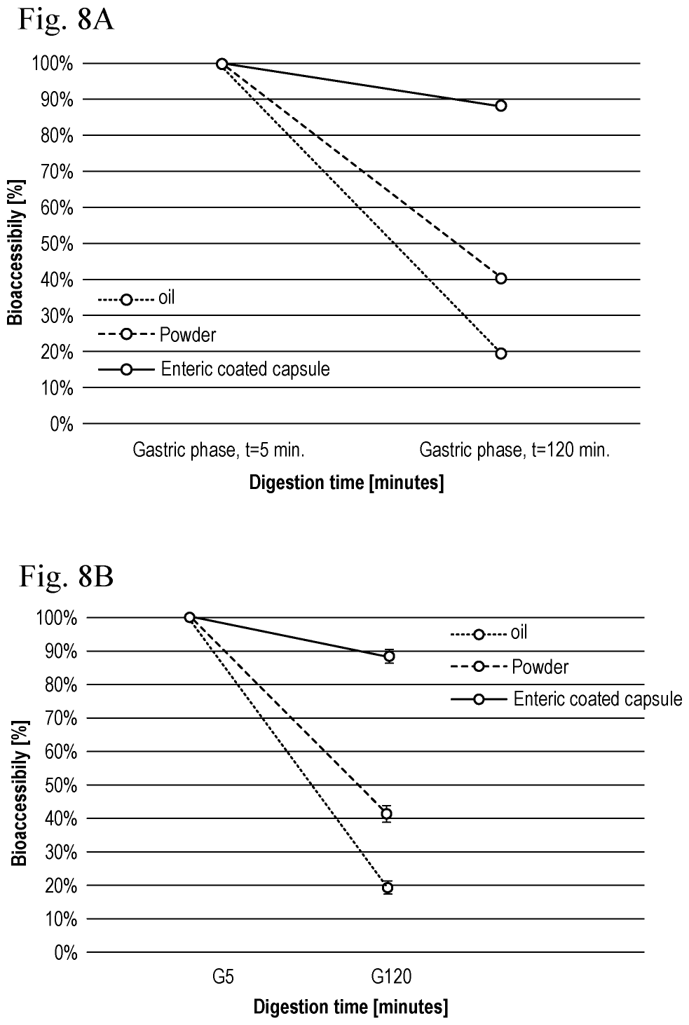

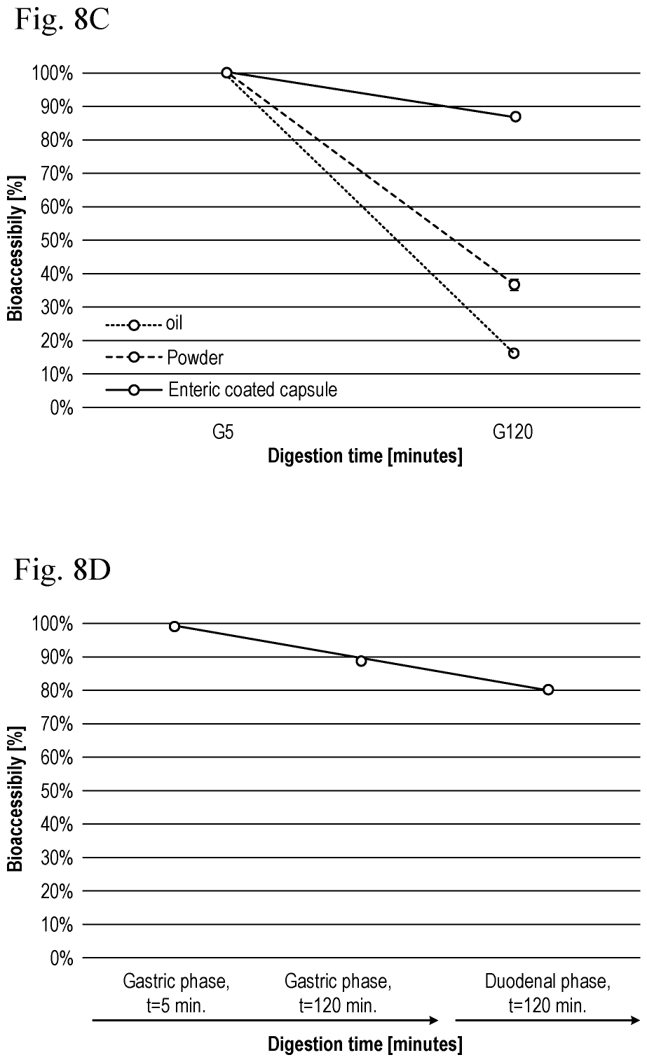

The bio-accessibility of Thymol and Carvacrol at the end of the gastric phase was 19%, 41% and 89% for the oil and powder forms and the powder in enteric coated capsules, respectively, suggesting significant differences between various types of compositions. Similar results were obtained for the separate actives. For Thymol for example, the bio-accessibility was 16%, 37% and 87%, respectively. The results are shown in FIGS. 8A-8C. The bio-accessibility of the powder in enteric coated capsules at the end of the duodenal phase was 79% (for both actives). The result are shown in FIG. 8D. The bio-accessibility of Carvacrol was 78% and Thymol 97%.

Conclusions

The results suggest that the protective effect of the powder compositions can be further enhanced by the addition of functional coating, thereby increasing even further their gastric and duodenal bio-accessibility.

Overall, the invention provides a highly relevant pharmaceuticals platform for formulating poorly water-soluble APIs to achieve improved oral bioavailability and bio-accessibility of incorporated actives.

### Example 7: Compositions Incorporated into PVA Films

7.1 Compositions Incorporated into a Sublingual Patch

The experiment explored application of the technology to PVA sublingual films. Powders containing 30-50% oil were reconstituted in TDW to 5% (w/w). PVA solution (4.5%) was prepared from PVA powder (86-89 hydrolyzed PVA) in TDW. PVA solution was mixed with the nanoemulsion in proportions of 4% and 0.5%, respectively. Samples (3 g, ×6 samples) were casted into aluminum mold and dried at 38° C. for 24 h. Some samples included flavoring agents. Samples' specifications are detailed in Table 16.

TABLE 16
Specifications of the sample

| | Nanoemulsion | Actual PVA | Sample | Dry |
|---|---|---|---|---|
| #sample | addition (g) | conc. | size (g) | weight (g) |
| 1 | | 8.0% | 2.5 | 0.20 |
| 2 | 2.1 | 7.6% | 4.2 | 0.34 |
| 3 | 2.1 | 7.3% | 4.2 | 0.32 |
| 4 | 2.1 | 6.9% | 4.2 | 0.31 |
| 5 | 2.1 | 6.6% | 4.2 | 0.28 |
| 6 | 2.1 | 6.3% | 4.2 | 0.30 |

All samples produced films, the differences in shape can be attributed to different wetting properties. Table 17 shows comparison between the actual dry weight and theoretical weight, suggesting a complete evaporation of water during drying. The nanoemulsion was uniformly dispersed across the films.

TABLE 17
Estimates of the actual and theoretical weight

| | Nano-particles | | |
|---|---|---|---|
| PVA theoretical | theoretical | Total theoretical | Actual dry |
| content (g) | content (g) | dry compounds (g) | weight (g) |
| 0.20 | 0.000 | 0.20 | 0.20 |
| 0.32 | 0.008 | 0.33 | 0.34 |
| 0.31 | 0.017 | 0.32 | 0.32 |
| 0.29 | 0.025 | 0.32 | 0.31 |
| 0.28 | 0.034 | 0.31 | 0.28 |
| 0.26 | 0.042 | 0.31 | 0.30 |

Selected samples (N=3) were dissolved in 50 ml TDW at 37° C. for 20-40 min. Sample 6 (dry weight 0.15 g) was analyzed for oil content, yielding about 0.017 g oil—83.6% of the theoretical content. The produced film (1*1 cm², ˜100 μm thick) was placed under the tongue measuring the time to complete dissolution.

The results suggest that the powder of the invention was suitable for formulation in polymeric films. The solid particles were evenly fixed in the polymerized film to create a solid-in-solid dispersion. Upon dissolution, the particles were completely released from the PVA matrix. Overall, a sub-lingual film provides an attractive approach for oral and transmucosal delivery of certain type of lipophilic APIs.

7.2 Compositions Incorporated into a Dermal and Transdermal Patch

Powders containing 30-50% oil were reconstituted in TDW to 0.5% (w/w). PVA solution (8%) and PVA/nanoemulsion mix were prepared as above, casted into aluminum mold and dried. Samples' specifications were similar (see Table 16). The produced film (2*1 cm², ˜100 μm thick) was dissolved in TDW at 37° C. and analyzed for oil content. Estimates of particles size before and after release from the film are show in Table 18.

TABLE 18
Estimates of particle size

| | Average particle size in | Average particle size |
|---|---|---|
| #sample | the pre-formulation | after release from PVA |
| 1 | 187 nm | 207 nm |
| 2 | 187 nm | 210 nm |
| 3 | 187 nm | 208 nm |
| 4 | 187 nm | 214 nm |
| 5 | 187 nm | 202 nm |
| 6 | 187 nm | 202 nm |

Nanometric particle size has a significant impact on the surface area of API and its permeation rate through biological membranes. In view of that, the finding that the particle size was maintained in the PVA formulations is particularly important; this is despite the exposure to polar environment (PVA film), temperature and drying. Upon drying, the solid particles were evenly fixed in the polymerized film to create a solid-in-solid dispersion. Stability of this structure can be attributed to the unique nanoparticulate nature of the present compositions. Upon dissolution, the particles were completely released from the polymer.

Overall, the results suggest that the present powder compositions can be incorporated into pharmaceutical dosage forms such as dermal films, thus providing an attractive innovative approach to dermal and transdermal delivery of actives. The natural humidity of the skin causes the film to dissolve slowly, thereby slowly releasing the nanoparticulate lipophilic actives embedded in the film until complete dissolution of the film and permeation of actives through the epidermal layers into the circulation. All these make dermal patches a particularly advantageous dosage form for prolonged delivery of actives through the skin.

### Example 8: Enhanced Permeability Through the Skin

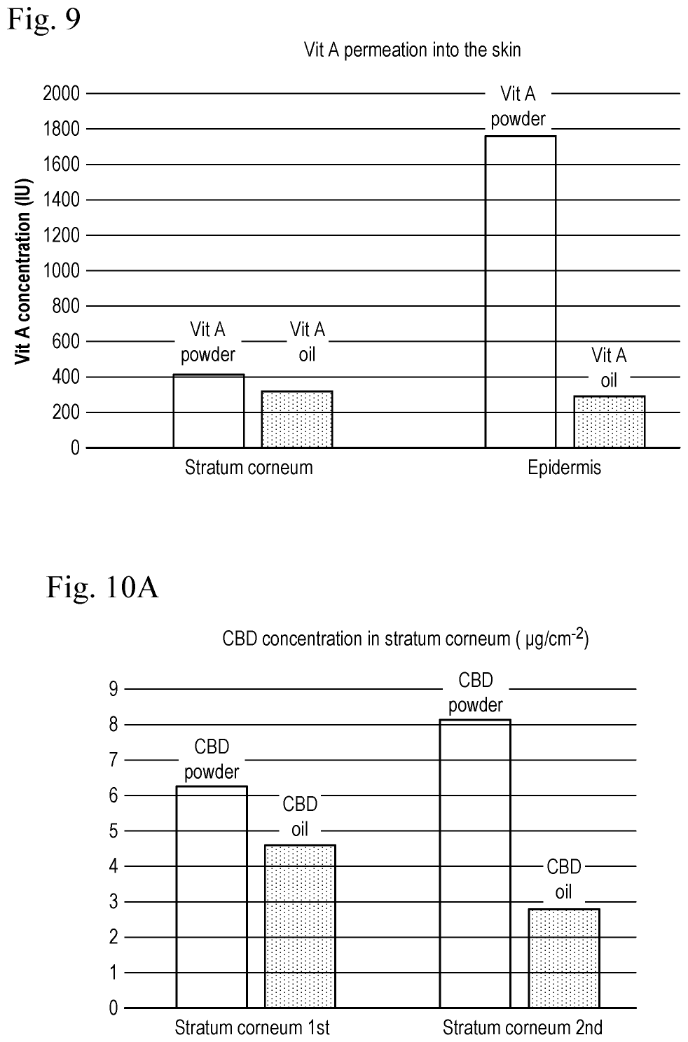

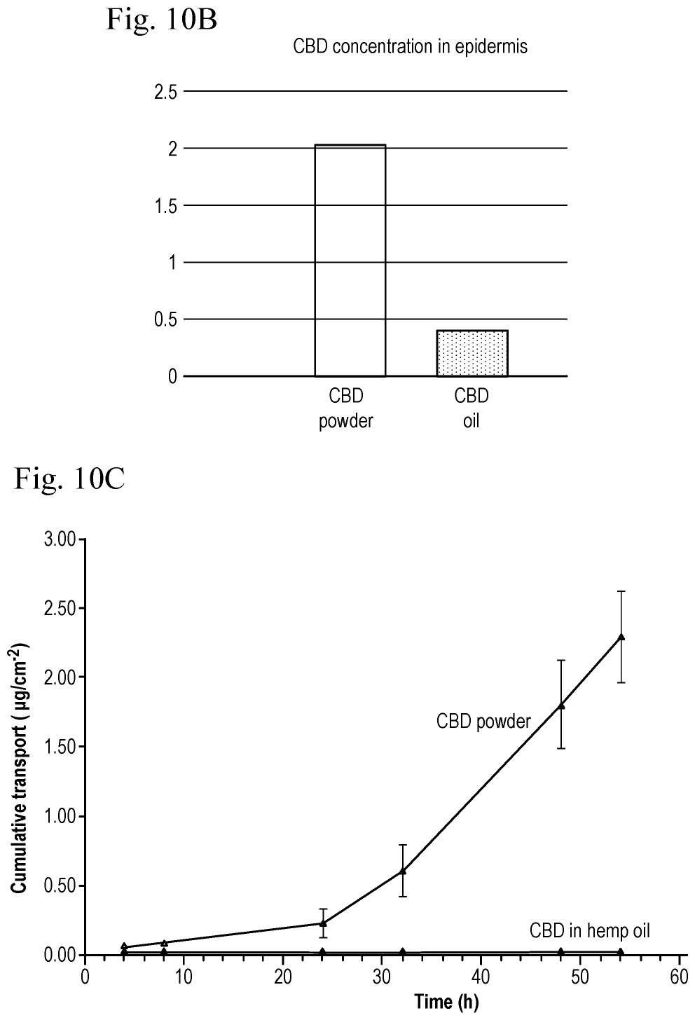

Permeability through the skin was studied with compositions containing Vitamin A and CBD comparing the respective powder and oil forms in ex vivo model of human skin. The results are shown in FIG. 9 and FIGS. 10A-10C.

The results suggest that the powder compositions of the invention have a significantly enhanced permeation through the various layers of human skin compared to the respective oil forms. For vitamin A for example, the permeability through the full-thickness of human skin was 6-fold higher for the powder compositions than for the respective oil forms (FIG. 9). For CBD, the permeability of the powder form was higher through the 1St outermost layer of the stratum corneum, and about 4-fold higher through the 2nd layer of the stratum corneum (FIG. 10A), yielding about 10-fold higher concertation of API in the epidermis overall (FIG. 10B) and significantly higher rate cumulative transport of API into the deeper layers of the skin (FIG. 10C).

### Example 9: Compositions in the from of Mist (Nebulizer)

An attractive method of administration, especially for certain types of APIs, is via an inhalation device or a nebulizer. To that end, the powder compositions were loaded into a nebulizer for home-use and the product was analyzed for particle size and other characteristic properties. Powder containing 30-50% oil (example of lipophilic API) was dissolved in TDW to the concentration of 20% to produce nanoemulsion, which was diluted to 10%, 4%, 2% and 1%. Samples' specifications are detailed in Table 19.

TABLE 19
Measurements of particle size

| | | | | | | Run | Residual |
|---|---|---|---|---|---|---|---|
| | Conc | | API | | DDW | time | amount |
| Nanoemulsion | (%) | API source | amount | units | (ml) | (min) | (g) |
| A | 20 | LL-C:H | 4 | g | 20 | 8:37 | 0.39 |
| | | 1:10 5-1-20 | | | | | |
| A | 20 | LL-C:H | 4 | g | 20 | 7:10 | 0.71 |
| | | 1:10 5-1-20 | | | | | |
| B | 10 | Nanoemulsion A | 10 | ml | 10 | 7:53 | 0.49 |
| C | 4 | Nanoemulsion B | 8 | ml | 12 | 7:30 | 0.49 |
| D | 2 | Nanoemulsion C | 10 | ml | 10 | 12:25 | 0.54 |
| E | 1 | Nanoemulsion D | 10 | ml | 10 | 8:43 | 0.60 |
| Water | 0 | NA | NA | NA | 2 | 12:00 | 0.56 |

Samples of the nanoemulsions (2 ml) and water control were loaded into the device. Nanoemulsion A was subjected to two runs for comparison. Upon activating the device, the mist was clearly visible for the recorded period. Residual nanoemulsion was visible on the device walls. The inhalation cup was weighed on a lab scale (0.01 g precision) before and after each test and residual weight was calculated.

The residual amount ranged from 0.39 to 0.71 gr. (17.7%-32.2%), with the average of 0.54 g. (about 25.4%). The nanoemulsion s.g ranged from 1.01-1.08 g/ml, depending on the concentration of the nanoemulsion, with the average residual amount similar to the distilled water.

Overall, the results suggest that reconstituted powder compositions can be used in nebulizers to produce a particulate nanometric material in the form of mist that is inhalable into to the respiratory tract. The effectiveness of mist production from the nanoemulsion was equivalent to water.

### Example 10: Compositions with Antibiotics

The study investigated the potential of encapsulating clarithromycin, an antibiotic against Pseudomonas aeruginosa, into the compositions of the invention. P. aeruginosa is the primary pathogen in the lungs of cystic fibrosis patients. This strain of bacteria is known for its ability to form biofilm, on biotic and abiotic surfaces, which makes it particularly resistant to host immune defenses and current antibiotic therapies. Clarithromycin, a new semisynthetic macrolide, is lipophilic molecule that exhibits a broad spectrum of antimicrobial activity against Gram-positive and -negative aerobes.

Compositions with clarithromycin were prepared by cold physical process. Powder clarithromycin compositions were dissolved in water to obtaining nanoemulsion. The control free clarithromycin was dissolved in 1% DMSO. Emulsion dried using lyophilization. Samples were tested in three independent experiments, with triplicate in each experiment. Particle size was measured using DLS. MIC data (minimum inhibition concentrations) of the formulated and free antibiotic were compared.

The results showed that powder compositions with the antibiotic retained its characteristic physical properties, including the nanometric particles size of about 180 nm (average diameter). Specifications of the nanoemulsion and the MIC data are summarized in Tables 20 and 21.

TABLE 20
Measurements of particle size

| Clarithromycin powder | Fine grant white powder |
|---|---|
| composition | |
| Particle size (nanoemulsion) | 150-200 nm |
| Excipients | Disaccharide, polysaccharide, |
| | natural emulsifier |
| pH (nanoemulsion) | 4.4 |
| Time to dissolution | <90 |
| Water content | <2 |

TABLE 21
MIC in the powder and oil compositions

| Powder composition with | |
|---|---|
| clarithromycin | Free clarithromycin |
| 0.03125%. | 0.0625% |

The results show that P. aeruginosa, a highly resistant strain, was more susceptible to the powder composition with the antibiotic than to the free form, with MIC 0.03125% mg/liter for the powder composition compared to 0.0625% for the free form (P<0.001). In other words, the results suggest that the effective dose of clarithromycin for achieving significant inhibition (50%) of P. aeruginosa the growth and expansion is lower for the powder compositions with clarithromycin compared to the free form of the same antibiotic.

Overall, the result show significantly increased susceptibility of P. aeruginosa to the powder compositions with clarithromycin (50%, P<0.05), thus proving the applicability of the present technology to enhance the efficacy of known lipophilic antibiotics against pathogenic bacteria, including highly resistant strains.

The results further suggest that the powder compositions may have the ability to disrupt and/or enhance the permeability of active through the bacterial biofilm. The powder compositions are essentially dispersed emulsifier-coated negatively charged lipid droplets. The present findings of increase efficacy of the drug can be explained by (1) the small particle size provides benefits for penetration of the drug and its accumulation in the bacterial biofilm; (2) the negatively charged nanoparticles are generally known to penetrate more easily into the biofilms; (3) diffusion coefficient depends on drug interaction with the EPS bacterial matrix constructing the biofilm.

In other words, the powder clarithromycin compositions have the potential to enhance absorption and accumulation of antibiotic actives in microbial biofilms, most likely due to the improved solubility of the emulsified lipid particles. Thus, the present technology provides a new platform for formulation of lipophilic antibiotics and develop-ment of new antimicrobial agents and delivery systems targeting microbial biofilms.

### Example 11: Formulations in Micronized Sugar Particles

### 11.1 Micronized Sugar Particles

Using the present technology, an example formulation of micronized sugar was prepared from sucrose, maltodextrin, sugar ester (SP30) and Theobroma oil. The amounts and the proportions of ingredients are detailed in Table 22. An example protocol of the production process is detailed further below.

TABLE 22
Amounts and concentrations of ingredients

| | Total | Concentration in the dry |
|---|---|---|
| Ingredient | amount (gr)* | formulation (% w/w) |
| Sucrose | 610 | 61 |
| Maltodextrin | 150 | 15 |
| Sugar ester (SP30) | 40 | 4 |
| Theobroma oil | 200 | 20 |
| Added water (DDW) | 2200 | NR |

*Total dry weight of all ingredients: 1000 gr

Essential steps in the process of making the formulation:
i. Sucrose and maltodextrin were mixed with DDW.
ii. Sugar ester (Sp30) was added, the solution was heated to 50° C. complete dissolution of ingredients.
iii. Theobroma oil was added, the solution was homogenized to produce uniform emulsion.
iv. The emulsion was fed to High Pressure Microfluidizer (4 bar, 16,000 PSI ×3 cycles), yielding nanodrops in the size range of about 100 nm-200 nm.
v. The nanoemulsion was frozen (−30° C.) and lyophilized until completely dry (about 2 days at 0.04 mBar). Alternatively, the frozen nanoemulsion was spray dried at about 190° C.

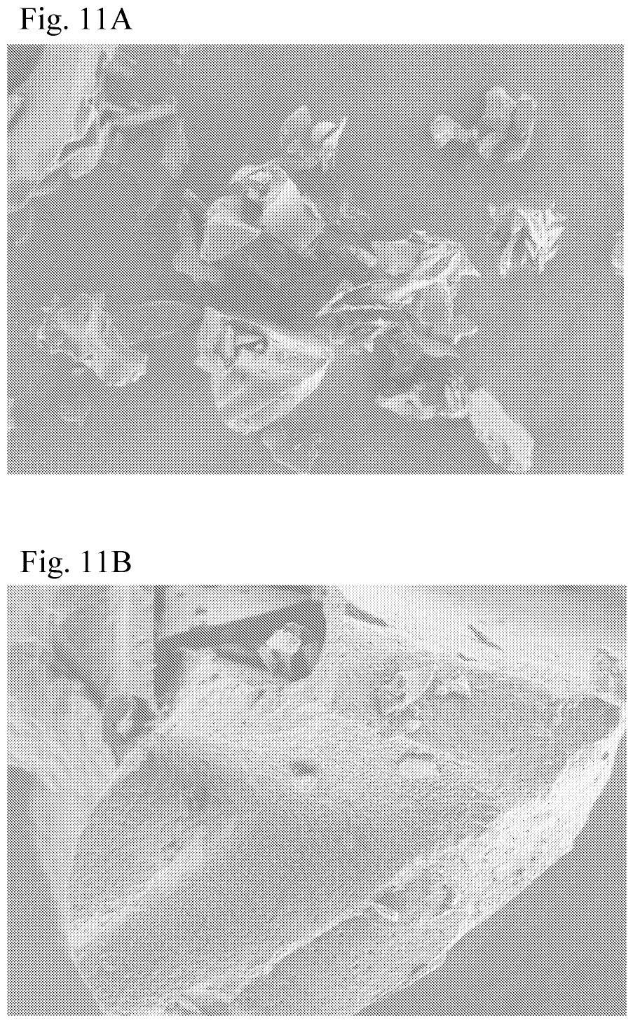

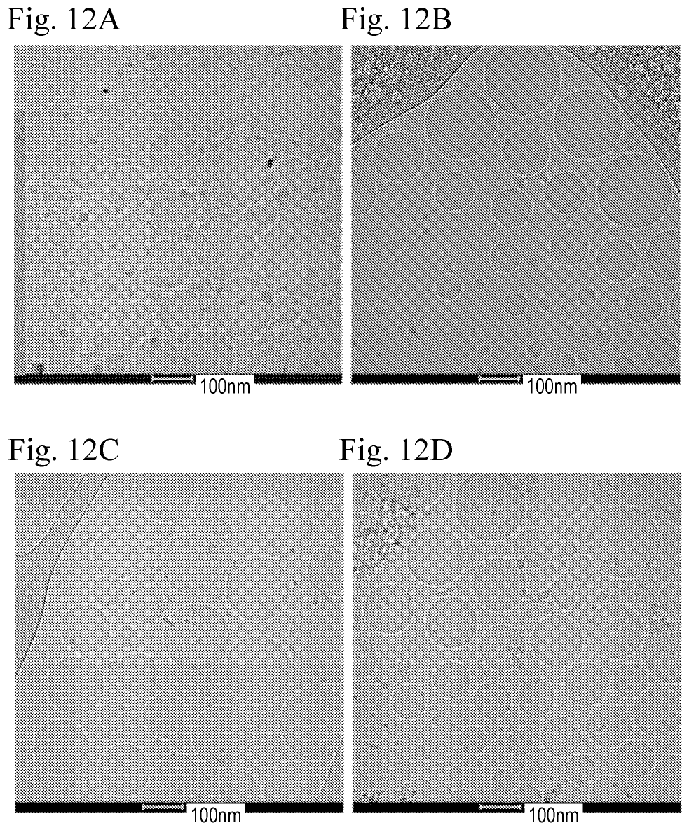

The powder product was analyzed by Scanned Electron Microscope (SEM). Images of the product in FIGS. 11A-1B show a smooth finely granulated sugar particles with size in the range of 20-50 μm. Overall, the results show that the sugar powder of the invention was relatively uniform in terms of texture and size, with smooth and finely granulated particles below 50 μm.

### 11.2 Entrapment of Nanometric Oil Drops in the Sugar Particle

The sugar particles with vitamin E oil (example of lipophilic API) were analyzed using Cryogenic Transmission Electron Microscopy (cryo-TEM). Samples were prepared in Controlled Environment Vitrification System (CEVS) with humidity at saturation to prevent evaporation of volatiles and temperature of 25° C. The solution (1 drop) was placed on carbon-coated perforated polymer film supported on 200 mesh TEM grid. The drop was converted to a thin film (<300 nm) by removing excess solution. The grid cooled in liquid ethane at −183° C. Cryo-TEM imaging was performed on Thermo-Fisher Talos F200C at 200 kV. Micrographs were recorded by Thermo-Fisher Falcon camera (4k×4k resolution). Samples were examined in TEM nanoprobe mode using volta phase plates. Imaging was performed at low dose mode and acquired by TEM TIA software.

Images of cryo-TEM sections in FIGS. 10A-10D show a population of smooth surfaced spherical nano-droplets with the average size in the range of about 80-150 nm, which is entrapped in the sugar particle.

### ANNEX A

A1. Classes of therapeutic agents relevant to the present compositions
Analgesics including non-narcotic and narcotic analgesics
Antacids
Antianxiety Drugs
Antiarrhythmics
Antibacterial agents
Antibiotics including naturally occurring, synthetic, broad-spectrum antibiotics
Anticoagulants and Thrornbolytics for arterial or venous thrombosis
Anticonvulsants
Antidepressants including mood-lifting antidepressants: tricyclics, monoamine oxidase inhibitors, and SSRIs
Antidiarrheals including antidiarrheal preparations and drugs that slow down the contractions of the bowel muscles
Antiemetics
Antifungals including infections that affect hair, skin, nails, mucous membranes
Antihistamines
Antihypertensives including diuretics, beta-blockers, calcium channel blocker, ACE (angiotensin-converting enzyme) inhibitors
Anti-Inflammatories
Antineoplastics
Antipsychotics Also major tranquilizers
Antipyretics
Antivirals including treatment and temporary protection against viral infections
Barbiturates (see sleeping drugs).
Beta-Blockers
Bronchodilators
Cold Cures in relations to aches, pains, and fever that accompany a cold
Corticosteroids in the context of immunosuppression, malignancies or deficiency disorders
Cough Suppressants including narcotic and non-narcotic suppressants
Cytotoxics as antineoplastics and also as immunosuppressives
Decongestants
Diuretics
Expectorant
Hormones: including synthetic equivalents and natural hormone extracts
Hypoglycemics (Oral)
Immunosuppressives
Laxatives
Muscle Relaxants including those that relieve muscle spasm and minor tranquilizers
Sedatives
Sex Hormones (Female) including those used for menstrual and menopausal disorders, oral contraceptives, and also for treating female and male cancers.
Sex Hormones (Male) including those used for male hormonal deficiency in hypopituitarism or disorders of the testes, also for treating cancer, and anabolic steroids
Sleeping Drugs
Tranquilizer including minor and major tranquilizers
Vitamins
   A2. Nutrient rich oils relevant to the present compositions

Major Pharmaceutically Acceptable Oils
Coconut oil, an oil high in saturated fat
Corn oil, an oil with little odor or taste
Cottonseed oil, an oil low in trans-fats
Canola oil, (a variety of rapeseed oil)
Olive oil
Palm oil, the most widely produced tropical oil
Peanut oil (ground nut oil)
Safflower oil
Sesame oil, including cold pressed light oil and hot-pressed darker oil
Soybean oil, produced as a byproduct of processing soy meal
Sunflower oil

Other Pharmaceutically Acceptable Oils
Almond oil
Cashew oil,
Hazelnut oil
Macadamia oil, has no trans-fats, and a good balance omega-3/omega-6
Pecan oil
Pistachio oil
Walnut oil

Nutrient Rich Oils
Amaranth oil, high in squalene and unsaturated fatty acids
Apricot oil
Argan oil, a food oil from Morocco
Artichoke oil, extracted from the seeds of Cynara cardunculus
Avocado oil
Babassu oil, a substitute for coconut oil
Ben oil, extracted from the seeds of Moringa oleifera
Borneo tallow nut oil, extracted from the fruit of Shorea
Buffalo gourd oil, extracted from the seeds of Cucurbita foetidissima
Carob pod oil (Algaroba oil)
Coriander seed oil
False flax oil made of the seeds of Camelina sativa
Grape seed oil
Hemp oil, a high quality food oil
Kapok seed oil
Lallemantia oil, extracted from the seeds of Lallemantia iberica
Meadowfoam seed oil, highly stable with over 98% long-chain fatty acids
Mustard oil (pressed)
Okra seed oil, extracted from the seed of Hibiscus esculentus
Perilla seed oil, high in omega-3 fatty acids
Pequi oil, extracted from the seeds of Caryocar brasiliensis
Pine nut oil, an expensive food oil from pine nuts
Poppyseed oil
Prune kernel oil, a gourmet cooking oil.
Pumpkin seed oil, a specialty cooking oil
Quinoa oil, similar to corn oil
Ramtil oil, pressed from the seeds of Guizotia abyssinica (Niger pea)
Rice bran oil
Tea oil (Camellia oil)
Thistle oil, pressed from the seeds of Silybum marianum.
   A3. Other substances relevant to the micronized sugar formulations

Natural Sugars
Beet sugar, white and granulated sugar
Cane sugar, white refined or brown sugar
Brown sugar, granulated cane sugar that has molasses (dark and light brown)
Demerara sugar, a type of raw cane sugar
Fructose, fruit sugar twice as sweet as refined cane sugar
Fruit sweetener (liquid and solid) made from grape juice concentrate blended with rice syrup
Jaggery (palm sugar, gur), made from the reduced sap of either the sugar palm or the palmyra palm
Maple sugar, much sweeter than white sugar and has fewer calories
Muscovado (Barbados) sugar, a raw cane sugar similar to brown sugar
Piloncillo (panela, panocha), another type of a raw cane sugar
Rock sugar (Chinese rock sugar), a lightly caramelized cane sugar
Sucanat: juice from organically grown sugarcane turned into granular sugar
Turbinado sugar, raw cane sugar crystals derived from sugarcane
White refined sugar (granulated sugar, table sugar, sucrose) derived from sugarcane or sugar beets

Edible Polysaccharides
Starch, generally a polymer consisting of two amylose (normally 20-30%) and amylopectin (normally 70-80%) primarily found in cereal grains and tubers like corn (maize), wheat, potato, tapioca, and rice
Kaempferia rotunda and Curcuma xanthorrhiza essential oils that are enriched in cassava starch-based polysaccharide
Maltodextrin, a polysaccharide produced from vegetable starch
Alginate, a naturally occurring anionic polymer obtained from brown seaweed, also used in various pharmaceutical preparations such as gaviscon, bisodol, and asilone
Carrageenans, water-soluble polymers with a linear chain of partially sulfated galactans
Pectins, a group of plant-derived polysaccharides
Agars, hydrophilic colloids that have the ability to form reversible gels
Chitosan, a promising group of natural polymers with characteristics such as biodegradability, chemical inertness, biocompatibility, high mechanical strength
Gums, edible-polymer preparations used for their texturizing capabilities
Certain cellulose derivative forms, predominantly four are used in the food industry: hydroxypropyl cellulose (HPC), hydroxypropyl methylcellulose (HPMC), carboxymethylcellulose (CMC), or methylcellulose (MC).

Food Emulsifiers
lecithin and lecithin derivatives
glycerol fatty acid esters
hydroxycarboxylic acid and fatty acid esters
lactylate fatty acid esters
polyglycerol fatty acid esters
ethylene or propylene glycol fatty acid esters
ethoxylated derivatives of monoglycerides

Natural and Nature-Identical Colorants Allowed in the EU and the USA
Curcumin (Turmeric
Riboflavin
Cochineal, Cochineal extract, carminic acid, carmines
Chlorophyll(in)s copper complexes chlorophyll(in)s
Caramel
Vegetable carbon
Carrot oil, β-carotene
Annatto, bixin, norbixin
Paprika extract
Lycopene
β-Apo-8′-carotenal
Ethyl ester of β-apo-8′-carotenoic acid
Lutein
Canthaxanthin
Beetroot red
Anthocyanins
Cottonseed flour
Vegetable juice
Saffron

Acidulants and other preservatives
Lactic acid, acetic acid and other acidulants, alone or in conjunction with other preservatives such as sorbate and benzoate
Malic and tartaric (tartric) acids
Citric acid
Ascorbic acid/vitamin C, isoascorbic isomer, erythorbic acid and their salts

Lipophilic Food Preservatives
Benzoic acid in the form of its sodium salt
Sorbic acid and potassium sorbate, specifically for mold and yeast inhibition
Lipophilic arginine esters, a more recent group of compounds

1. A solid water-dispersible composition of matter comprising at least one sugar, at least one polysaccharide and at least one surfactant and at least one lipophilic active pharmaceutical ingredient (API),
the composition comprises a plurality of micrometric particles each comprising a plurality of lipophilic nanospheres with an average size in the range of about 50 nm to about 900 nm, the at least one lipophilic API is contained in the micrometric particles and is distributed inside and/or or inside and outside the lipophilic nanospheres at predetermined proportions, thereby providing an improved delivery of the at least one lipophilic API,
wherein the composition has
a loading capacity of the at least one lipophilic API up to about 50% (w/w) relative to total weight, and/or
an encapsulation capacity of the at least one lipophilic API in the range of between about 70% and about 98%.

2-8. (canceled)

9. The composition of claim 8, wherein the micrometric particles have an average size between about 10 μm and to about 300 μm.

10. (canceled)

11. The composition of claim 1, wherein the size of lipophilic nanospheres is substantially maintained upon dispersion in water.

12. The composition of claim 1, wherein the at least one lipophilic API is dissolved in at least one pharmaceutically acceptable oil.

13-15. (canceled)

16. The composition of claim 12, wherein the at least one pharmaceutically acceptable oil is a natural oil, a synthetic oil, a modified natural oil, or a combination thereof.

17. The composition of claim 12, wherein the at least one pharmaceutically acceptable oil selected from acylglycerols, mono- (MAG), di- (DAG) and triacylglycerols (TAG), medium-chain triglycerides (MCT), long chain triglycerides (LCT), saturated or unsaturated fatty acids.

18. (canceled)

19. The composition of claim 1, wherein the at least one sugar is selected from oligo, mono-, di-saccharides and polyols, optionally trehalose, sucrose, mannitol, lactitol and lactose.

20. The composition of claim 1, wherein the at least one polysaccharide is selected from maltodextrin and carboxymethyl cellulose (CMC).

21. The composition of claim 1, wherein the at least one surfactant is selected from ammonium glycyrrhizinate, pluronic F-127 or pluronic F-68.

22. The composition of claim 1, wherein the at least one surfactant is selected from a natural emulsifier, a monoglyceride, a diglycerine, a glycolipid, a lecithin, a fatty alcohol, a fatty acid or a mixture thereof.

23. The composition of claim 1, wherein the at least one surfactant is a sucrose fatty acid ester (sugar ester).

24. (canceled)

25. (canceled)

26. The composition of claim 1, wherein the at least one lipophilic API is selected from enzyme inhibitors, receptor antagonists or agonists, proton-pump inhibitors, ion-channel inhibitors, and/or reuptake inhibitors.

27. The composition of claim 1, wherein the at least one lipophilic API is selected from antibiotics, antifungal agents, antiviral agents, neuroleptics, analgesics, hormones, anti-inflammatory drugs, non-steroidal anti-inflammatory drugs, anti-rheumatic, drugs anticoagulants, beta-blockers, diuretics, anti-hypertension drugs, anti-atherosclerosis drugs, antidiabetics, anti-asthmatic drugs, decongestant and/or cold medicines.

28. (canceled)

29. The composition of claim 1, wherein the improved delivery of the at least one lipophilic API comprises an improved oral bioavailability of the at least one lipophilic API in plasma or at least one tissue, said at least one tissue being at least one tissue of the central nervous system (CNS), least one lymphatic tissue, or at least one tissue of a part of the GI lumen, or the liver tissue.

30. (canceled)

31. The composition of claim 1, wherein the improved delivery of the at least one lipophilic API comprises an improved bio-accessibility of the at least one lipophilic API into at least a part of the gastrointestinal (GI) tract or at least one tissue in the GI tract.

32. The composition of claim 1, further comprising a pharmaceutically acceptable carrier and/or excipient.

33. The composition of claim 32, the composition being adapted for oral, sublingual, buccal administrations, or rectal, topical, dermal, or transdermal administrations, or inhalation or nebulization.

34-39. (canceled)

40. A dosage form comprising a therapeutically effective amount of the composition of claim 1, and further optionally comprising a coating, a shell, or a capsule.

41-51. (canceled)

52. A method for improving oral bioavailability and/or bio-accessibility of at least one lipophilic API in a subject in need thereof, the method comprising administering to the subject a therapeutically effective amount of
a solid water-dispersible composition of matter comprising at least one sugar, at least one polysaccharide and at least one surfactant and at least one lipophilic active pharmaceutical ingredient (API),
the composition comprises a plurality of micrometric particles each comprising a plurality of lipophilic nanospheres with an average size in the range of about 50 nm to about 900 nm, the at least one lipophilic API is contained in the micrometric particles and is distributed inside or inside and outside the lipophilic nanospheres at predetermined proportions, thereby providing an improved delivery of the at least one lipophilic API,
wherein the composition has
a loading capacity of the at least one lipophilic API up to about 50% (w/w) relative to total weight, and/or
an encapsulation capacity of the at least one lipophilic API in the range of between about 70% and about 98%.

53-55. (canceled)

56. A method for treating or alleviating a disorder or a condition that can be remedied by treatment with least one lipophilic API in a subject in need thereof, the method comprises administering to the subject a therapeutically effective amount of
a solid water-dispersible composition of matter comprising at least one sugar, at least one polysaccharide and at least one surfactant and at least one lipophilic active pharmaceutical ingredient (API),
the composition comprises a plurality of micrometric particles each comprising a plurality of lipophilic nanospheres with an average size in the range of about 50 nm to about 900 nm, the at least one lipophilic API is contained in the micrometric particles and is distributed inside or inside and outside the lipophilic nanospheres at predetermined proportions, thereby providing an improved delivery of the at least one lipophilic API,
wherein the composition has
a loading capacity of the at least one lipophilic API up to about 50% (w/w) relative to total weight, and/or
an encapsulation capacity of the at least one lipophilic API in the range of between about 70% and about 98%.

57-77. (canceled)
